(19) United States
(12) Patent Application Publication
(10) Pub. No.: US 2024/0041991 A1
(43) Pub. Date: Feb. 8, 2024

(54) STABLE LIQUID DISPERSINB COMPOSITIONS

(71) Applicant: KANE BIOTECH INC., Winnipeg (CA)

(72) Inventors: NANDADEVA YAKANDAWALA, Winnipeg (CA); GORDON GUAY, Winnipeg (CA)

(73) Assignee: ONTARIO POWER GENERATION INC., Winnipeg, MB (CA)

(21) Appl. No.: 17/802,343

(22) PCT Filed: Feb. 25, 2021
(86) PCT No.: PCT/CA2021/050231

§ 371 Date: Aug. 25, 2022

Related U.S. Application Data
(60) Provisional application No. 62/981,269, filed on Feb. 25, 2020.

(51) Int. Cl. A61K 38/47; A61K 47/10; A61K 47/26

(52) U.S. Cl. CPC A61K 38/47; A61K 47/10; A61K 47/26

(57) ABSTRACT

Citrate buffer, a polyol, a polymer, a salt, a preservative are used individually or in combination in a liquid coating or film composition with DispersinB to stabilize the DispersinB at an ambient or higher temperature. The polyol may comprise sorbitol, glycerol, propylene glycol, isomalt, erythritol, or maltitol. The polymer may comprise poloxamer 407, polyvinyl alcohol, gelatin, cellulose, hydroxyethyl cellulose, carboxymethyl cellulose, or polyvinylpyrrolidone. The salt may comprise NaCl, Na₂SO₄, NH₄Cl, KCl, KNO₃, or K₂SO₄. The preservative may comprise ethylenediaminetetraacetic acid (EDTA), levulinic acid, or anisic acid.

### CROSS-REFERENCE TO RELATED APPLICATION

This patent application claims the benefit of U.S. Provisional Patent Application No. 62/981,269, filed Feb. 25, 2020 which is incorporated by reference in its entirety herein.

### FIELD OF INVENTION

The present invention relates to DispersinB stabilizing liquid coating or film compositions and use of particular compounds in the DispersinB compositions.

### BACKGROUND

DispersinB is an enzyme that is naturally produced by a periodontal disease-associated oral bacterium, Aggregatibacter actinomycetemcomitans. It specifically hydrolyses the glycosidic linkages of poly-beta 1,6N-acetylglucosamine (PNAG) leading to destabilization of biofilm structure and exposing biofilm-embedded bacteria. Purified recombinant DispersinB is shown to be active against diverse mammalian pathogens. In particular, PNAG is produced by a wide range of bacteria and fungi and is a key component in biofilm formation.

DispersinB cleaves PNAG, inhibiting bacterial adhesion and disperses the biofilm. This is especially useful for treating wounds and otic infections, which can become chronic due to the persistent nature of the bacterial biofilms. Once the biofilm is dispersed the bacteria can be eradicated and the infection can be remedied.

DispersinB composition should be stabilized to retain DispersinB's functional activity for a prolonged period of time. Improvement of both storage and/or shelf stability, and operational stability are important when developing and using DispersinB. Storage stability refers to retention of enzymatic activity over time, and operational stability relates to the retention of activity of an enzyme when in use. If a stabilized enzyme system is not used, excess of enzyme is generally required to compensate for expected loss of activity. However, enzymes are expensive to produce. A major challenge with DispersinB in solution is to maintain its enzymatic activity during manufacturing, formulation, shipping, handling and application and to avoid inactivation.

Purified DispersinB is very stable as lyophilized powder. However, DispersinB, like most of the biological enzymes, is sensitive to elevated temperatures and temperature variations, being susceptible to thermal denaturation. There is no known or published DispersinB liquid formulations that can be stored at room temperature or higher without losing enzymatic activity in a short period of time.

Therefore, the present inventors have found that liquid forms of the enzyme can be stored at refrigerated or lower temperatures, typically −20° C. For long-term storage at −20° C., the DispersinB solutions can include a phosphate buffer (with pH 5.9), and glycerol (50%) added to prevent cryo damage. DispersinB, in its known buffer and pH, is known to lose its enzymatic activity within one day at ambient temperature.

This rapid loss of DispersinB activity at room temperature in liquid form hinders its use in commercial products and restricts its versatility. Further, formulation, storage and transportation of DispersinB at low temperature tends to increase logistical issues and, consequently, increases cost. Since a loss of DispersinB enzymatic activity at body temperature is anticipated in soluble form, its potential therapeutic uses and application in humans and animals are generally restricted and untested.

A major technological challenge is to develop a DispersinB containing product that can be stored and transported at room temperature, and which also protects the DispersinB enzyme from thermal denaturation and helps to maintain high enzymatic activity.

It is also important to ensure stability of the DispersinB at human and animal body temperatures for reasonable periods of time in order to use it as therapeutic. To develop DispersinB into commercial products, other than as a lyophilized powder, it is highly desirable for the DispersinB to be stable at an ambient temperature or higher, and desirable for its enzymatic activity to be maintained for an extended period of time.

### SUMMARY OF INVENTION

In embodiments, the present invention provides uses of a citrate buffer in a liquid coating or film composition with DispersinB to stabilize the DispersinB. The present invention also provides liquid coating or film compositions thereof.

In other embodiments, the present invention provides uses of a polyol in a liquid coating or film composition with DispersinB to stabilize the DispersinB at an ambient or higher temperature. The polyol may comprise sorbitol, glycerol, propylene glycol, isomalt, erythritol, or maltitol. The present invention also provides liquid coating or film compositions thereof.

In other embodiments, the present invention provides uses of a polymer in a liquid coating or film composition with DispersinB to stabilize the DispersinB at an ambient or higher temperature. The polymer may comprise poloxamer 407, polyvinyl alcohol, gelatin, cellulose, hydroxyethyl cellulose, carboxymethyl cellulose, or polyvinylpyrrolidone. The present invention also provides liquid coating or film compositions thereof.

In other embodiments, the present invention provides uses of a salt in a liquid coating or film composition with DispersinB to stabilize the DispersinB at an ambient or higher temperature. The salt may comprise NaCl, Na₂SO₄, NH₄Cl, KCl, KNO₃, or K₂SO₄. The present invention also provides liquid coating or film compositions thereof.

In other embodiments, the present invention provides uses of a preservative in a liquid coating or film composition with DispersinB to sterilize the DispersinB at an ambient or higher temperature. The preservative may comprise ethylenediaminetetraacetic acid (EDTA), levulinic acid, or anisic acid. The present invention also provides liquid coating or film compositions thereof.

In other embodiments, the present invention provides uses of a polyol and a polymer in a liquid coating or film composition with DispersinB to stabilize the DispersinB at an ambient or higher temperature. The polyol may comprise sorbitol, and the polymer may comprise poloxamer 407. The present invention also provides liquid coating or film compositions thereof.

In other embodiments, the present invention provides uses of a polyol, a polymer, and a preservative in a liquid coating or film composition with DispersinB to stabilize the DispersinB at an ambient or higher temperature. The polyol may comprise sorbitol, the polymer may comprise poloxamer 407, and the preservative may comprise ethylenediaminetetraacetic acid (EDTA). The present invention also provides liquid coating or film compositions thereof.

The present invention encompasses any and all combinations of any of the polyols, polymers, salts, preservatives, and buffers described herein, for stabilization of DispersinB.

### BRIEF DESCRIPTION OF THE FIGURES

Exemplary embodiments are illustrated in referenced figures of the drawings. It is intended that the embodiments and figures disclosed herein are to be considered illustrative rather than restrictive.

FIG. 1 shows a bar graph illustrating the effect of phosphate buffer and citrate buffer on thermal stability and enzymatic activity of DispersinB.

FIG. 2 shows a bar graph illustrating the effect of sorbitol on thermal stability and enzymatic activity of DispersinB.

FIG. 3 shows a bar graph illustrating the effect of glycerol on thermal stability and enzymatic activity of DispersinB.

FIG. 4 shows a bar graph illustrating the effect of mannitol on thermal stability and enzymatic activity of DispersinB.

FIG. 5 shows a bar graph illustrating the effect of PEG on thermal stability and enzymatic activity of DispersinB.

FIG. 6 shows a bar graph illustrating the effect of propylene glycol on thermal stability and enzymatic activity of DispersinB.

FIG. 7 shows a bar graph illustrating the effect of xylitol on thermal stability and enzymatic activity of DispersinB.

FIG. 8 shows a bar graph illustrating the effect of inositol on thermal stability and enzymatic activity of DispersinB.

FIG. 9 shows a bar graph illustrating the effect of sorbitol and glycerol on thermal stability and enzymatic activity of DispersinB.

FIG. 10 shows a bar graph illustrating the effect of isomalt on thermal stability and enzymatic activity of DispersinB.

FIG. 11 shows a bar graph illustrating the effect of erythritol on thermal stability and enzymatic activity of DispersinB.

FIG. 12 shows a bar graph illustrating the effect of maltitol on thermal stability and enzymatic activity of DispersinB.

FIG. 13 shows a bar graph illustrating the effect of poloxamer 407 on thermal stability and enzymatic activity of DispersinB.

FIG. 14 shows a bar graph illustrating the effect of salts on thermal stability and enzymatic activity of DispersinB.

FIG. 15 shows a bar graph illustrating the effect of pH on thermal stability and enzymatic activity of DispersinB.

FIG. 16 shows a bar graph illustrating the effect of potassium sulfate on thermal stability and enzymatic activity of DispersinB.

FIG. 17 shows a bar graph illustrating the effect of levulinic acid on thermal stability and enzymatic activity of DispersinB.

FIG. 18 shows a bar graph illustrating the effect of anisic acid on thermal stability and enzymatic activity of DispersinB.

FIG. 19 shows a bar graph illustrating the effect of EDTA and phosphate on thermal stability and enzymatic activity of DispersinB.

FIG. 20 shows a bar graph illustrating the effect of EDTA and citrate on thermal stability and enzymatic activity of DispersinB.

FIG. 21 shows a bar graph illustrating the effect of sorbitol and poloxamer 407 on thermal stability and enzymatic activity of DispersinB.

FIG. 22 shows a bar graph illustrating the effect of 30% sorbitol, 5% poloxamer 407, 50 mM Citrate buffer (pH 5.9), 100 mM sodium chloride, 1% Levulinic acid, 0.3% anisic acid, and 0.1% EDTA on thermal stability and enzymatic activity of DispersinB.

FIG. 23 shows a bar graph illustrating the effect of 30% sorbitol, 5% poloxamer 407, 50 mM Citrate buffer (pH 5.9), 100 mM sodium chloride, 1% Levulinic acid, 0.3% anisic acid, and 0.1% EDTA on thermal stability and enzymatic activity of DispersinB.

FIG. 24 shows a bar graph illustrating the effect of 30% sorbitol, 5% poloxamer 407, 50 mM Citrate buffer (pH 5.9), 100 mM sodium chloride, 1% Levulinic acid, 0.3% anisic acid, and 0.1% EDTA on thermal stability and enzymatic activity of DispersinB.

FIG. 25 shows a bar graph illustrating the effect of 30% sorbitol, 5% PF127, 50 mM citrate buffer (pH 5.9), 100 mM sodium chloride, 1% levulinic acid, anisic acid, and 0.1% EDTA on thermal stability and enzymatic activity of DispersinB.

FIG. 26 shows a bar graph illustrating the effect of 30% sorbitol, 5% PF127, 50 mM citrate buffer (pH 5.9), 100 mM sodium chloride, 1% levulinic acid, anisic acid, and 0.1% EDTA on thermal stability and enzymatic activity of DispersinB.

### DETAILED DESCRIPTION

The inventors have found that a number of compounds, individually and in combination with one another, are useful as stabilizers in helping to maintain DispersinB in a liquid coating or film composition at room temperature or higher. In particular, the present subject matter relates to liquid DispersinB compositions comprising one or more of purified water, polyols, polymers, salts, a buffering system, and preservatives.

The examples and data below show the effects on the enzymatic activity of the DispersinB when particular compounds are combined in a liquid DispersinB composition.

### Effect of Buffer and DH on DispersinB

Traditionally, phosphate buffer is used as the standard buffer in liquid DispersinB compositions when they are placed in long-term storage at −20° C. Buffers were tested to determine whether they had an effect on the stability of DispersinB at ambient or higher temperatures.

Lyophilized DispersinB powder was dissolved in selected buffers (citrate, phosphate) of defined pH (5.5 -7.5). The DispersinB concentration was adjusted to 100 μg/ml. Salt concentration was maintained at 100 mM sodium chloride. DispersinB enzymatic activity was measured using β-N-Acetylglucosaminidase assay kit from Sigma (product code CS0780) in 96-well microtiter plate following the manufacturer's instructions. The enzymatic activity was presented as percentages in comparison to enzymatic activity of DispersinB in 100 mM Citrate buffer, 100 mM NaCl, pH 5.9, which was considered 100%.

Table 1 illustrates the effect of buffer and pH on the enzymatic activity of DispersinB. Enzymatic activity in citrate buffer with a pH of 5.9 was considered 100%. * indicates statistically significant (p<0.05) values in paired two tailed t-test, each treatment was compared with enzymatic activity of DispersinB in corresponding buffer of pH 5.9.

TABLE 1

| Buffer | Buffer | | % Enzyme | |
|---|---|---|---|---|
| system | strength | pH | activity | ±SD |
| Citrate | 100 mM | 5.5 | 72* | 10.15 |
| Citrate | 100 mM | 5.9 | 100 | 0.74 |
| Citrate | 100 mM | 6 | 76* | 5.27 |
| Citrate | 100 mM | 6.5 | 53* | 0.13 |
| Citrate | 100 mM | 7 | 35* | 0.52 |
| Citrate | 100 mM | 7.5 | 21* | 0.77 |
| Phosphate | 100 mM | 5.5 | 80* | 0.78 |
| Phosphate | 100 mM | 5.9 | 100 | 1.29 |
| Phosphate | 100 mM | 6 | 81* | 0.07 |
| Phosphate | 100 mM | 6.5 | 50* | 0.21 |
| Phosphate | 100 mM | 7 | 24* | 0.27 |
| Phosphate | 100 mM | 7.5 | 12* | 0.96 |

As demonstrated in Table 1 and FIG. 1, use of a citrate buffer results in as good, if not enhanced, enzymatic stability of DispersinB as compared to the use of a typical phosphate buffer, especially with a pH around 5 to 6.

The clinically relevant maintenance of DispersinB enzymatic activity, with the use of a citrate buffer indicates that citrate buffer may be used to stabilize DispersinB in liquid coating or film compositions.

Thus, in one aspect, the present invention provides a use of a citrate buffer with DispersinB in a liquid coating or film composition to stabilize the DispersinB at an ambient or higher temperature.

In an embodiment, the concentration of citrate buffer used is between 10 mM and 500 mM. In a preferred embodiment, the concentration of citrate buffer used is between 50 mM and 200 mM. In another preferred embodiment, the concentration of citrate buffer used is about 100 mM.

In the above embodiments of use of citrate buffer with DispersinB, the liquid coating or film composition may have a pH between 4 and 7.5. In a preferred embodiment, the pH of the liquid coating or film composition is between 4.6 and 6.5. In a further preferred embodiment, the pH of the liquid coating or film composition is between 5.5 and 5.9.

Thus, in another aspect, the present invention provides a liquid coating or film composition comprising citrate buffer and DispersinB.

In an embodiment, the concentration of citrate buffer in the liquid coating or film composition is between 10 mM and 500 mM. In a preferred embodiment, the concentration of citrate buffer in the liquid coating or film composition is between 50 mM and 200 mM. In another preferred embodiment, the concentration of citrate buffer in the liquid coating or film composition is about 100 mM.

In the above embodiments of the liquid coating or film composition with citrate buffer and DispersinB, the liquid coating or film composition may have a pH between 4 and 7.5. In a preferred embodiment, the pH of the liquid coating or film composition is between 4.6 and 6.5. In a further preferred embodiment, the pH of the liquid coating or film composition is between 5.5 and 5.9.

### Use of a Polvol with DispersinB

A number of polyols were tested to determine their effect on the thermal stability of DispersinB at an ambient and elevated temperatures, including sorbitol, glycerol, propylene glycol, isomalt, erythritol, and maltitol.

For each polyol, a DispersinB enzyme solution (100 μg/ml) was prepared in 50 mM citrate buffer (pH 5.9), 100 mM sodium chloride with the polyol. Samples from each formula was incubated at 3 different temperatures (42° C., 52° C. and 62° C.) for 3 hours. Following a 3 hour incubation, the samples were brought to room temperature for enzymatic activity assay.

DispersinB enzymatic activity was measured using β-N-Acetylglucosaminidase assay kit from Sigma (product code CS0780) in 96-well microtiter plate following the manufacturer's instructions. The data was represented as % enzymatic activity in comparison to enzyme activity of freshly made control sample that contained 100 μg/ml DispersinB in 50 mM citrate buffer (pH 5.9), 100 mM sodium chloride without polyol. Activity of the control sample was considered 100%. Two-tailed paired T-test was performed by to compare each polyol containing treatment with the treatment devoid of polyol. Treatment with probability values (p) less than 5% (0.05) was considered significant.

Of the polyols tested, sorbitol, glycerol, xylitol, and inositol exhibited temperature stabilizing effect on DispersinB at elevated temperatures, including 42° C., 52° C., and 62° C. Mannitol, xylitol, polyethylene glycol, and propylene glycol did not exhibit temperature stabilizing effects on DispersinB at elevated temperatures. Thus, in one aspect, the present invention provides a use of sorbitol, glycerol, xylitol, and/or inositol with DispersinB in a liquid coating or film composition, to stabilize the DispersinB at an ambient or higher temperature.

Sorbitol, C₆H₁₄O₆ or (2S,3R,4R,5R)-Hexane-1,2,3,4,5,6-hexol, is a sugar alcohol commonly obtained from the reduction of glucose. Sorbitol is commonly used as a sugar substitute to sweeten medications, candy, gums, and baked goods. The chemical structure of sorbitol is:

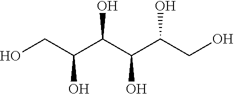 [SMILES: OC[C@@H](O)[C@@H](O)[C@H](O)[C@@H](O)CO]

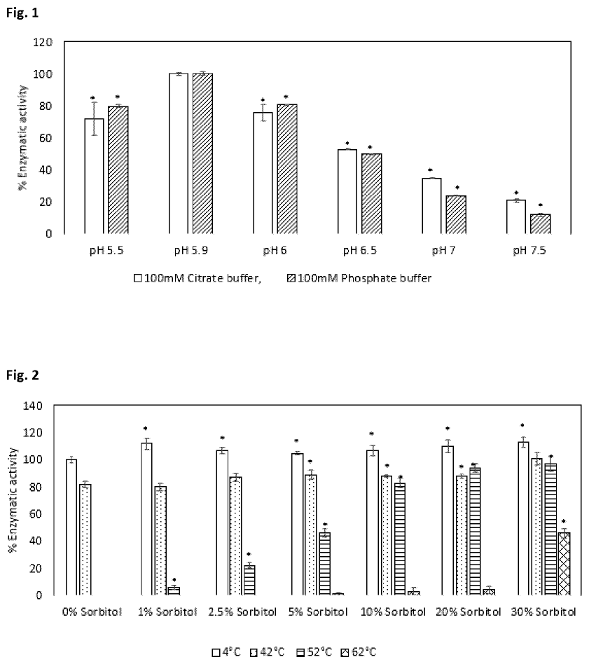

Table 2 and FIG. 2 illustrates the effect of sorbitol on thermal stability of DispersinB according to the test set out above. * indicates statistically significant (p<0.05) values in paired two tailed t-test, with each treatment compared with the standard treatment that contained 0% sorbitol.

TABLE 2

| | 4° C. | | 42° C. | | 52° C. | | 62° C. | |
|---|---|---|---|---|---|---|---|---|
| | Mean | SD | Mean | SD | Mean | SD | Mean | SD |
| Sorbitol 0% | 100 | 2.22 | 82 | 2.43 | — | — | — | — |
| Sorbitol 1% | 112* | 4.17 | 80 | 2.54 | 6* | 1.70 | — | — |
| Sorbitol 2.5% | 107* | 2.31 | 87 | 3.21 | 21* | 1.95 | — | — |
| Sorbitol 5% | 105* | 1.06 | 89* | 3.41 | 46* | 2.94 | 1* | 1.14 |
| Sorbitol 10% | 107* | 3.87 | 88* | 0.9 | 82* | 3.78 | 3* | 2.72 |
| Sorbitol 20% | 110* | 4.32 | 88* | 1.31 | 94* | 2.76 | 4* | 3.01 |
| Sorbitol 30% | 113* | 4.05 | 101* | 4.71 | 97* | 5.09 | 46* | 3.49 |

As demonstrated, DispersinB enzymatic activity largely remained around 100% when 10% to 30% of sorbitol by weight was added to the composition and incubated at 52° C. for 3 hours, especially when 20% and 30% of sorbitol by weight was added. In particular, notable DispersinB enzymatic activity remained when 30% of sorbitol by weight was added to the composition and incubated at 62° C. for 3 hours.

The clinically relevant maintenance of DispersinB enzymatic activity with the use of sorbitol indicates that sorbitol is useful in stabilizing DispersinB in liquid coating or film compositions at ambient or higher temperatures.

Thus, in one aspect, the present invention provides a use of sorbitol with DispersinB in a liquid coating or film composition, to stabilize the DispersinB at an ambient or higher temperature.

In an embodiment, the amount of sorbitol used is up to 50% of the composition by weight. In a preferred embodiment, the amount of sorbitol used is between 20% and 40% of the composition by weight. In another preferred embodiment, the amount of sorbitol used is between 25% and 35% of the composition by weight. In a further preferred embodiment, the amount of sorbitol used is about 30% of the composition by weight.

In another aspect, the present invention provides a liquid coating or film composition comprising sorbitol and DispersinB.

In an embodiment, the amount of sorbitol in the liquid coating or film composition is up to 50% of the composition by weight. In a preferred embodiment, the amount of sorbitol in the liquid coating or film composition is between 20% and 40% of the composition by weight. In another preferred embodiment, the amount of sorbitol in the liquid coating or film composition is between 25% and 35% of the composition by weight. In a further preferred embodiment, the amount of sorbitol in the liquid coating or film composition is about 30% of the composition by weight.

Glycerol, C₃H₈O₃ or (Propane-1,2,3-triol), also called glycerine or glycerin, is a polyol compound that is colorless, odorless, and viscous in liquid form. Since it is also sweet-tasting and non-toxic, glycerol is widely used as a sweetener and humectant in food and medications.

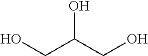

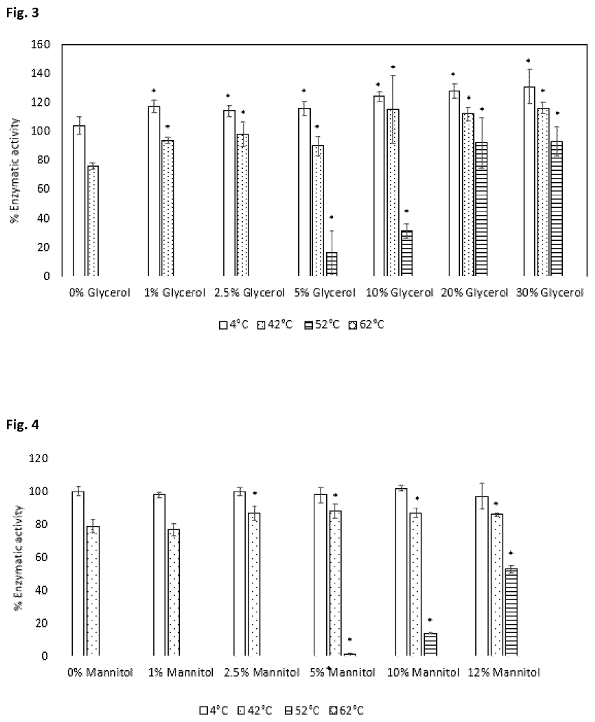

Table 3 and FIG. 3 illustrate the effect of glycerol on thermal stability of DispersinB according to the test set out above. * indicates statistically significant (p<0.05) values in paired two tailed t-test, with each treatment compared with the standard treatment that contained 0% glycerol.

TABLE 3

| | 4° C. | | 42° C. | | 52° C. | | 62° C. | |
|---|---|---|---|---|---|---|---|---|
| | Mean | SD | Mean | SD | Mean | SD | Mean | SD |
| Glycerol 0% | 104 | 6.08 | 76 | 2.28 | — | — | — | — |
| Glycerol 1% | 117* | 4.25 | 94* | 2.18 | — | — | — | — |
| Glycerol 2.5% | 114* | 3.93 | 98* | 8.44 | — | — | — | — |
| Glycerol 5% | 116* | 5.05 | 90* | 6.69 | 16* | 15.19 | — | — |
| Glycerol 10% | 124* | 3.34 | 115* | 23.73 | 31* | 4.91 | — | — |
| Glycerol 20% | 128* | 5.15 | 112* | 4.53 | 92* | 17.43 | — | — |
| Glycerol 30% | 131* | 11.83 | 116* | 3.94 | 93* | 9.66 | — | — |

As demonstrated, DispersinB enzymatic activity remained largely around 100 when at least 10% of glycerol was added to the composition and incubated at 42° C. for 3 hours. Notably, DispersinB enzymatic activity remained largely around 100% when 20% or 30% of glycerol by weight was added to the composition and incubated at 52° C. for 3 hours.

The clinically relevant maintenance of DispersinB enzymatic activity with the use of glycerol indicates that glycerol is useful in stabilizing DispersinB in liquid coating or film compositions at ambient or higher temperatures.

Thus, in one aspect, the present invention provides a use of glycerol with DispersinB in a liquid coating or film composition, to stabilize the DispersinB at an ambient or higher temperature.

In an embodiment, the amount of glycerol used is up to 50% of the composition by weight. In a preferred embodiment, the amount of glycerol used is between 20% and 40% of the composition by weight. In another preferred embodiment, the amount of glycerol used is between 25% and 35% of the composition by weight. In a further preferred embodiment, the amount of glycerol used is about 30% of the composition by weight.

In another aspect, the present invention provides a liquid coating or film composition comprising glycerol and DispersinB.

In an embodiment, the amount of glycerol in the liquid coating or film composition is up to 50% of the composition by weight. In a preferred embodiment, the amount of glycerol in the liquid coating or film composition is between 20% and 40% of the composition by weight. In another preferred embodiment, the amount of glycerol in the liquid coating or film composition is between 25% and 35% of the composition by weight. In a further preferred embodiment, the amount of glycerol in the liquid coating or film composition is about 30% of the composition by weight.

Mannitol, C₆H₁₄O₆, is a sugar alcohol often used in medications and as a sweetener in food. In medication, mannitol is used as a diuretic for people with acute (sudden) kidney failure, and in injections to reduce swelling and pressure inside the eye or around the brain. The chemical structure of mannitol is:

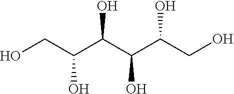 [SMILES: OC[C@@H](O)[C@@H](O)[C@H](O)[C@H](O)CO]

Table 4 and FIG. 4 illustrate the effect of mannitol on thermal stability of DispersinB according to the test set out above. * indicates statistically significant (p<0.05) values in paired two tailed t-test, with each treatment compared with the standard treatment that contained 0% mannitol.

TABLE 4

| | 4º C. | | 42° C. | | 52° C. | | 62° C. | |
|---|---|---|---|---|---|---|---|---|
| | Mean | SD | Mean | SD | Mean | SD | Mean | SD |
| 0% Mannitol | 100 | 2.85 | 79 | 4.19 | — | — | — | — |
| 1% Mannitol | 98 | 1.49 | 77 | 3.63 | — | — | — | — |
| 2.5% Mannitol | 100 | 2.46 | 87* | 4.46 | — | — | — | — |
| 5% Mannitol | 98 | 4.73 | 88* | 4.36 | 1* | 1.11 | — | — |
| 10% Mannitol | 102 | 1.46 | 87* | 2.69 | 14* | 0.42 | — | — |
| 12% Mannitol | 97 | 7.71 | 86* | 0.95 | 53* | 2.2 | — | — |

As demonstrated, mannitol did not meaningfully contribute to thermal stability of DispersinB at elevated temperatures (52° C. and 62° C.). Mannitol was ineffective in significantly stabilizing DispersinB at temperatures 42° C. as compared to 0% mannitol or 52° C. at concentrations 5% or above.

Polyethylene glycol, C₂ₙH₄ₙ₊₂Oₙ₊₁, is a polyether compound with a number of applications, from industrial manufacturing to medicine. Also referred to as PEG, polyethylene oxide (PEO) or polyoxyethylene (POE), the chemical structure of polyethylene glycol is:

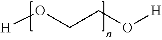

Table 5 and FIG. 5 illustrate the effect of PEG on thermal stability of DispersinB according to the test set out above. * indicates statistically significant (p<0.05) values in paired two tailed t-test, with each treatment compared with the standard treatment that contained 0% polyethylene glycol.

TABLE 5

| | 4° C. | | 42° C. | | 52° C. | | 62° C. | |
|---|---|---|---|---|---|---|---|---|
| | Mean | SD | Mean | SD | Mean | SD | Mean | SD |
| PEG 0% | 103 | 12.26 | 76 | 2.28 | — | — | — | — |
| PEG 1% | 112 | 10.09 | 103* | 2.14 | — | — | — | — |
| PEG 2.5% | 108 | 2.42 | 106* | 2.68 | — | — | — | — |
| PEG 5% | 106 | 1.66 | 103* | 2.55 | — | — | — | — |
| PEG 10% | 106 | 1.56 | 87* | 4.96 | — | — | — | — |
| PEG 20% | 96 | 2.55 | 88* | 3.02 | — | — | — | — |
| PEG 30% | 109 | 7.04 | 71* | 6.87 | — | — | — | — |
| PEG 40% | 90 | 9.94 | — | — | — | — | — | — |

As demonstrated, polyethylene glycol, even as high as 30%, did not contributed to thermal stability of DispersinB at elevated temperatures (52° C. and 62° C.). Polyethylene glycol was ineffective in stabilizing DispersinB at temperatures above 42° C. and showed destabilizing effect even at 42° C. at concentrations above 5% .

Propylene glycol, C₃H₈O₂, is an organic compound that is generally a viscous, colorless, faintly sweet liquid. Propylene glycol is miscible with a broad range of solvents, including water, acetone, and chloroform. The chemical structure of propylene glycol is:

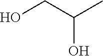

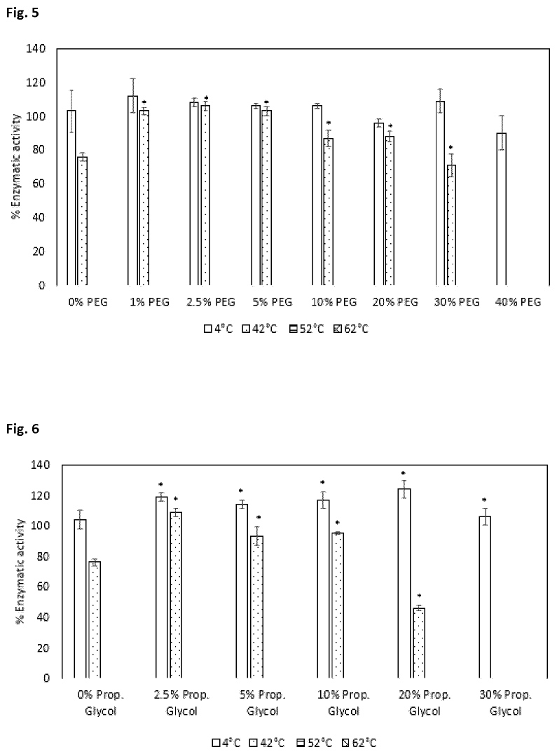

Table 6 and FIG. 6 illustrate the effect of propylene glycol on thermal stability of DispersinB according to the test set out above. * indicates statistically significant (p<0.05) values in paired two tailed t-test, with each treatment compared with the standard treatment that contained 0% propylene glycol.

TABLE 6

| | 4° C. | | 42° C. | | 52° C. | | 62° C. | |
|---|---|---|---|---|---|---|---|---|
| | Mean | SD | Mean | SD | Mean | SD | Mean | SD |
| Prop. Glycol 0% | 104 | 6.01 | 76 | 2.28 | — | — | — | — |
| Prop. Glycol 2.5% | 119* | 2.49 | 109* | 2.69 | — | — | — | — |
| Prop. Glycol 5% | 114* | 2.84 | 93* | 6.11 | — | — | — | — |
| Prop. Glycol 10% | 117* | 5.32 | 95* | 0.61 | — | — | — | — |
| Prop. Glycol 20% | 124* | 5.42 | 46* | 1.63 | — | — | — | — |
| Prop. Glycol 30% | 106 | 5.56 | — | — | — | — | — | — |

As demonstrated, propylene glycol, even as high as 30%, did not contributed to thermal stability of DispersinB B at elevated temperatures (52° C. and 62° C.). Propylene glycol was ineffective in stabilizing DispersinB at temperatures above 42° C. and showed destabilizing effect even at 42° C. at concentrations above 5%.

Xylitol, C₅H₁₂O₅, is a polyalcohol and a sugar alcohol. It is used as a sweetening agent, an allergen, a hapten, a human metabolite, an algal metabolite, a Saccharomyces cerevisiae metabolite and a mouse metabolite. The chemical structure of xylitol is:

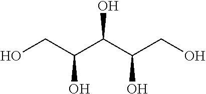

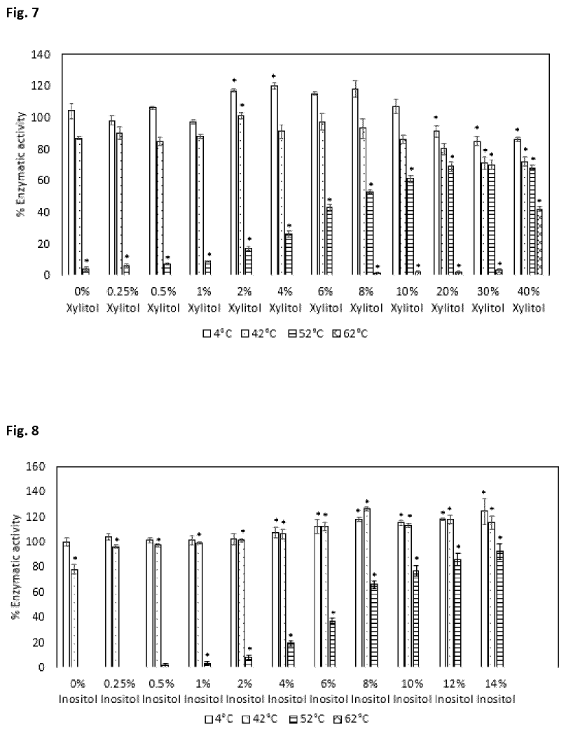

Table 7 and FIG. 7 illustrate the effect of xylitol on thermal stability of DispersinB. * indicates statistically significant (p<0.05) values in paired two tailed t-test, with each treatment compared with the standard treatment that contained 0% xylitol.

TABLE 7

| | 4° C. | | 42° C. | | 52° C. | | 62° C. | |
|---|---|---|---|---|---|---|---|---|
| | Avg | ±SD | Avg | ±SD | Avg | ±SD | Avg | ±SD |
| 0% Xylitol | 104 | 4.59 | 87 | 1.22 | 4 | 1.13 | — | — |
| 0.25% Xylitol | 98 | 3.08 | 90 | 3.9 | 6 | 1.17 | — | — |
| 0.5% Xylitol | 106 | 1.07 | 85 | 2.71 | 7 | 0.74 | — | — |
| 1% Xylitol | 97 | 1.39 | 88 | 1.12 | 9* | 0.16 | — | — |
| 2% Xylitol | 117* | 1.23 | 101* | 1.87 | 17* | 1.22 | — | — |
| 4% Xylitol | 120* | 1.76 | 91 | 4.24 | 26* | 1.75 | — | — |
| 6% Xylitol | 115 | 0.91 | 97 | 5.29 | 43* | 2.27 | — | — |
| 8% Xylitol | 118 | 5.08 | 93 | 6.36 | 53* | 0.97 | 1* | 0.93 |
| 10% Xylitol | 107 | 4.44 | 86 | 2.55 | 61* | 2.4 | 2* | 0.35 |
| 20% Xylitol | 91* | 3.72 | 80 | 3.49 | 69* | 2.97 | 2* | 0.67 |
| 30% Xylitol | 85* | 2.75 | 71* | 3.93 | 70* | 2.97 | 3* | 1.11 |
| 40% Xylitol | 86* | 1.23 | 72* | 3 | 68* | 1.56 | 42* | 1.4 |

As demonstrated, the reduction in DispersinB enzymatic activity was diminished when 20% to 40% of xylitol was added to the composition and incubated at 52° C. for three hours. In particular, some DispersinB enzymatic activity remained when 40% of xylitol by weight was added to the composition and incubated at 62° C. for 3 hours.

The clinically relevant maintenance of DispersinB enzymatic activity, with the use of xylitol indicates that xylitol is useful in stabilizing DispersinB in liquid coating or film compositions at ambient or higher temperatures.

Thus, in one aspect, the present invention provides a use of xylitol with DispersinB in a liquid coating or film composition, to stabilize the DispersinB at ambient or higher temperatures.

In an embodiment, the amount of xylitol used is up to 60% of the composition by weight. In a preferred embodiment, the amount of xylitol used is between 30% and 50% of the composition by weight. In a further preferred embodiment, the amount of xylitol used is about 40% of the composition by weight.

In another aspect, the present invention provides a liquid coating or film composition comprising xylitol and DispersinB.

In an embodiment, the amount of xylitol in the liquid coating or film composition is up to 60% of the composition by weight. In a preferred embodiment, the amount of xylitol in the liquid coating or film composition is between 30% and 50% of the composition by weight. In a further preferred embodiment, the amount of xylitol in the liquid coating or film composition is about 40% of the composition by weight.

Inositol, C₆H₁₅O₁₅P₃, is a carbocyclic sugar that is commonly found in brain and other mammalian tissues. It is a sugar alcohol with half the sweetness of sucrose (table sugar). The chemical structure of inositol is:

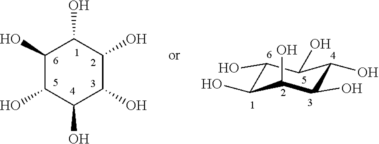

Table 8 and FIG. 8 illustrate the effect of inositol on thermal stability of DispersinB. * indicates statistically significant (p<0.05) values in paired two tailed t-test, with each treatment compared with the standard treatment that contained 0% inositol.

TABLE 8

| | 4° C. | | 42° C. | | 52° C. | | 62° C. | |
|---|---|---|---|---|---|---|---|---|
| | Mean | SD | Mean | SD | Mean | SD | Mean | SD |
| 0% Inositol | 100 | 3.25 | 78 | 3.78 | — | — | — | — |
| 0.25% Inositol | 104 | 2.66 | 96* | 1.39 | — | — | — | — |
| 0.5% Inositol | 101 | 2 | 97* | 1.07 | 1* | 2.41 | — | — |
| 1% Inositol | 101 | 3.68 | 99* | 1.1 | 3* | 1.45 | — | — |
| 2% Inositol | 102 | 4.75 | 101* | 1.29 | 8* | 1.85 | — | — |
| 4% Inositol | 107* | 4.02 | 106* | 3.66 | 19* | 1.78 | — | — |
| 6% Inositol | 112* | 5.68 | 112* | 3.2 | 37* | 2.38 | — | — |
| 8% Inositol | 118* | 1.36 | 126* | 1.96 | 66* | 2.81 | — | — |
| 10% Inositol | 115* | 1.7 | 113* | 1.98 | 77* | 4.27 | — | — |
| 12% Inositol | 118* | 1.01 | 118* | 3.43 | 86* | 4.91 | — | — |
| 14% Inositol | 124* | 10.14 | 115 | 5.46 | 92* | 6.19 | — | — |

As demonstrated, the reduction in DispersinB enzymatic activity was significantly diminished when up to 14% of inositol was added to the composition and incubated at 42° C. for three hours. Notably, the reduction in DispersinB enzymatic activity was significantly diminished when 10 to 14% of inositol was added to the composition and incubated at 52° C. for three hours, especially when 14% of inositol was added.

The clinically relevant maintenance of DispersinB enzymatic activity, with the use of inositol indicates that this compound is useful in stabilizing DispersinB in liquid coating or film compositions at ambient or higher temperatures.

Thus, in one aspect, the present invention provides a use of inositol with DispersinB in a liquid coating or film composition, to stabilize the DispersinB at an ambient or higher temperature.

In an embodiment, the amount of inositol used is up to 25% of the composition by weight. In a preferred embodiment, the amount of inositol used is between 10% and 20% of the composition by weight. In a further preferred embodiment, the amount of inositol used is about 14% of the composition by weight.

In another aspect, the present invention provides a liquid coating or film composition comprising inositol and DispersinB.

In an embodiment, the amount of inositol in the liquid coating or film composition is up to 25% of the composition by weight. In a preferred embodiment, the amount of inositol in the liquid coating or film composition is between 10% and 20% of the composition by weight. In a further preferred embodiment, the amount of inositol in the liquid coating or film composition is about 14% of the composition by weight.

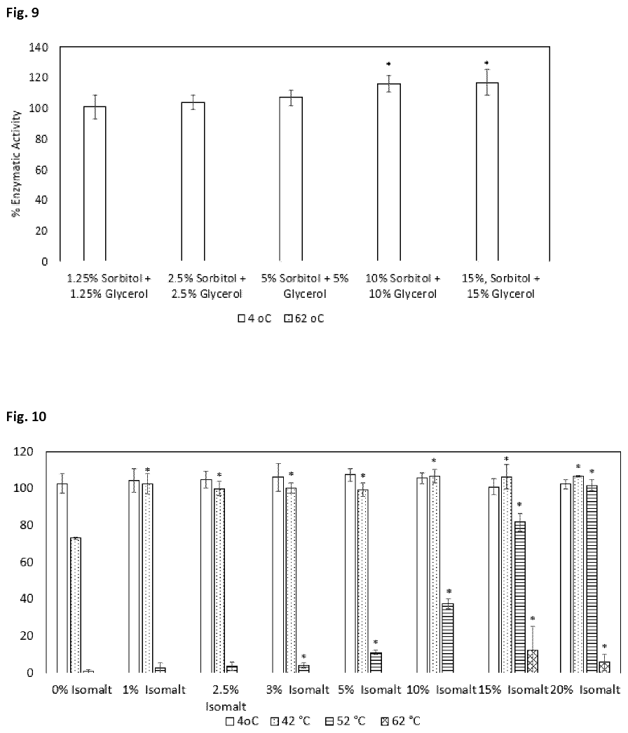

Sorbitol was also tested in combination with glycerol. Table 9 and FIG. 9 illustrate the effect of sorbitol and glycerol on thermal stability and enzymatic activity of DispersinB. * indicates statistically significant (p<0.05) values in paired two tailed t-test, with each treatment compared with the standard treatment that contained 0% glycerol and sorbitol. Significant variations were not found.

TABLE 9

| | 4° C. | | 62° C. | |
|---|---|---|---|---|
| | Mean | SD | Mean | SD |
| 15%, Sorbitol + 15% Glycerol | 117* | 8.21 | — | — |
| 10% Sorbitol + 10% Glycerol | 116* | 5.372 | — | — |
| 5% Sorbitol + 5% Glycerol | 107 | 5.113 | — | — |
| 2.5% Sorbitol + 2.5% Glycerol | 104 | 4.456 | — | — |
| 1.25% Sorbitol + 1.25% Glycerol | 101 | 7.52 | — | — |

Isomalt, C₁₂H₂₄O₁₁ or (2R,3R,4R,5R)-6-[[(2S,3R,4S,5S,6R)- 3,4,5-Trihydroxy-6-(hydroxymethyl)-2-tetrahydropyranyl]oxy]hexane-1,2,3,4,5-pentol, is a sugar alcohol. Isomalt is commonly used as a sugar substitute to sweeten medications, candy, gums, and baked goods. The chemical structure of isomalt is:

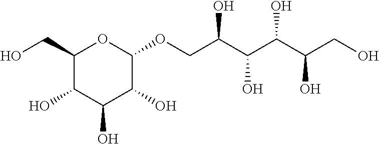 [SMILES: OC[C@@H](O)[C@@H](O)[C@H](O)[C@H](O)CO[C@H]1O[C@H](CO)[C@@H](O)[C@H](O)[C@H]1O]

Table 10 and FIG. 10 illustrate the effect of isomalt on thermal stability of DispersinB according to the test set out above. * indicates statistically significant (p<0.05) values in paired two tailed t-test, with each treatment compared with the standard treatment that contained 0% isomalt.

TABLE 10

| | 4° C. | | 42° C. | | 52° C. | | 62° C. | |
|---|---|---|---|---|---|---|---|---|
| | Mean | ±SD | Mean | ±SD | Mean | ±SD | Mean | ±SD |
| 0% Isomalt | 103 | 5.21 | 73 | 0.56 | 1 | 0.74 | 0 | 0 |
| 1% Isomalt | 105 | 6.46 | 102* | 5.53 | 2 | 2.80 | 0 | 0 |
| 2.5% Isomalt | 105 | 4.65 | 100* | 3.82 | 3 | 2.32 | 0 | 0 |
| 3% Isomalt | 106 | 7.59 | 100* | 2.92 | 4* | 1.29 | 0 | 0 |
| 5% Isomalt | 107 | 3.46 | 99* | 3.66 | 11* | 1.22 | 0 | 0 |
| 10% Isomalt | 106 | 2.97 | 107* | 3.65 | 37* | 2.73 | 0 | 0 |
| 15% Isomalt | 101 | 4.32 | 106* | 6.68 | 82* | 4.77 | 12* | 13.08 |
| 20% Isomalt | 102 | 2.59 | 107* | 0.56 | 102* | 2.97 | 6* | 4.23 |

As demonstrated, DispersinB enzymatic activity largely remained around 100% when up to 20% of isomalt by weight was added to the composition and incubated at 4° C. to 42° C. for 3 hours.

The clinically relevant maintenance of DispersinB enzymatic activity with the use of isomalt indicates that isomalt is useful in stabilizing DispersinB in liquid coating or film compositions at ambient or higher temperatures.

Thus, in one aspect, the present invention provides a use of isomalt with DispersinB in a liquid coating or film composition, to stabilize the DispersinB at an ambient or higher temperature.

In an embodiment, the amount of isomalt used is up to 20% of the composition by weight. In a preferred embodiment, the amount of isomalt used is between 1% and 20% of the composition by weight. In another preferred embodiment, the amount of isomalt used is between 5% and 10% of the composition by weight. In a further preferred embodiment, the amount of isomalt used is about 1% of the composition by weight.

In another aspect, the present invention provides a liquid coating or film composition comprising isomalt and DispersinB.

In an embodiment, the amount of isomalt in the liquid coating or film composition is up to 20% of the composition by weight. In a preferred embodiment, the amount of sorbitol in the liquid coating or film composition is between 1% and 15% of the composition by weight. In a further preferred embodiment, the amount of sorbitol in the liquid coating or film composition is about 1% of the composition by weight.

Erythritol, C₄H₁₀O₄ or (2R,3S)-Butane-1,2,3,4-tetrol, is a sugar alcohol. Erythritol is commonly used as a sugar substitute to sweeten medications, candy, gums, and baked goods. The chemical structure of erythritol is:

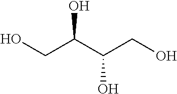

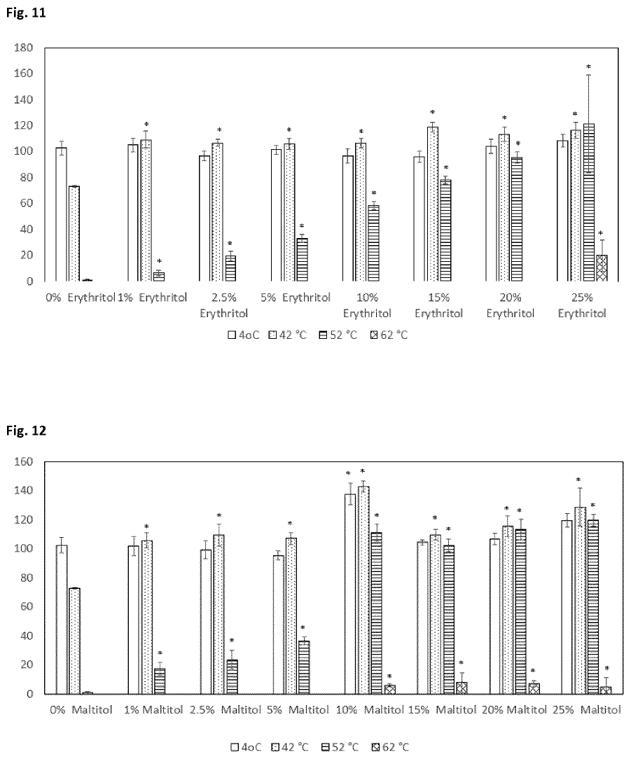

Table 11 and FIG. 11 illustrate the effect of erythritol on thermal stability of DispersinB according to the test set out above. * indicates statistically significant (p<0.05) values in paired two tailed t-test, with each treatment compared with the standard treatment that contained 0% erythritol.

TABLE 11

| | 4° C. | | 42° C. | | 52° C. | | 62° C. | |
|---|---|---|---|---|---|---|---|---|
| | Mean | ±SD | Mean | ±SD | Mean | ±SD | Mean | ±SD |
| 0% Erythritol | 103 | 5.21 | 73 | 0.56 | 1 | 0.74 | 0 | 0 |
| 1% Erythritol | 105 | 5.27 | 109* | 6.25 | 6* | 2.32 | 0 | 0 |
| 2.5% Erythritol | 97 | 3.88 | 107* | 2.96 | 20* | 3.87 | 0 | 0 |
| 5% Erythritol | 101 | 3.43 | 106* | 4.13 | 33* | 3.13 | 0 | 0 |
| 10% Erythritol | 97 | 5.34 | 107* | 3.83 | 58* | 2.87 | 0 | 0 |
| 15% Erythritol | 96 | 4.12 | 119* | 3.75 | 78* | 2.87 | 0 | 0 |
| 20% Erythritol | 104 | 5.52 | 113* | 5.71 | 95* | 4.18 | 0 | 0 |
| 25% Erythritol | 109 | 4.87 | 117* | 6.20 | 121* | 37.52 | 20* | 12.19 |

As demonstrated, DispersinB enzymatic activity largely remained near 100% when up to 25% of erythritol by weight was added to the composition and incubated at 4° C. for 3 hours. DispersinB enzymatic activity increased over 100% when up to 25% of erythritol by weight was added to the composition and incubated at 42° C. for 3 hours. . DispersinB enzymatic activity increased over 100% when 25% of erythritol by weight was added to the composition and incubated at 52° C. for 3 hours.

The clinically relevant maintenance of DispersinB enzymatic activity with the use of erythritol indicates that erythritol is useful in stabilizing DispersinB in liquid coating or film compositions at ambient or higher temperatures.

Thus, in one aspect, the present invention provides a use of erythritol with DispersinB in a liquid coating or film composition, to stabilize the DispersinB at an ambient or higher temperature.

In an embodiment, the amount of erythritol used is up to 25% of the composition by weight. In a preferred embodiment, the amount of erythritol used is between 1% and 20% of the composition by weight. In another preferred embodiment, the amount of erythritol used is between 5% and 10% of the composition by weight. In a further preferred embodiment, the amount of erythritol used is about 25% of the composition by weight.

In another aspect, the present invention provides a liquid coating or film composition comprising erythritol and DispersinB.

In an embodiment, the amount of erythritol in the liquid coating or film composition is up to 25% of the composition by weight. In a preferred embodiment, the amount of erythritol in the liquid coating or film composition is between 1% and 15% of the composition by weight. In a further preferred embodiment, the amount of erythritol in the liquid coating or film composition is about 25% of the composition by weight.

Maltitol, C₁₂H₂₄O₁₁ or 4-O-α-D-Glucopyranosyl-D-glucitol, is a sugar alcohol. Maltitol is commonly used as a sugar substitute to sweeten medications, candy, gums, and baked goods. The chemical structure of maltitol is:

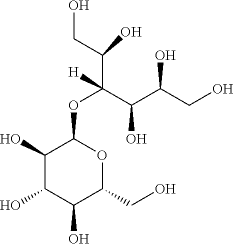 [SMILES: OC[C@@H](O)[C@@H](O[C@H]1O[C@H](CO)[C@@H](O)[C@H](O)[C@H]1O)[C@H](O)[C@@H](O)CO]

Table 12 and FIG. 12 illustrate the effect of maltitol on thermal stability of DispersinB according to the test set out above. * indicates statistically significant (p<0.05) values in paired two tailed t-test, with each treatment compared with the standard treatment that contained 0% maltitol.

TABLE 12

| | 4° C. | | 42° C. | | 52° C. | | 62° C. | |
|---|---|---|---|---|---|---|---|---|
| | Mean | ±SD | Mean | ±SD | Mean | ±SD | Mean | ±SD |
| 0% Maltitol | 103 | 5.21 | 73 | 0.56 | 1 | 0.74 | 0 | 0 |
| 1% Maltitol | 102 | 6.53 | 106* | 5.21 | 17* | 4.26 | 0 | 0 |
| 2.5% Maltitol | 99 | 6.51 | 110* | 7.63 | 24* | 6.17 | 0 | 0 |
| 5% Maltitol | 95 | 3.04 | 107* | 4.07 | 37* | 2.69 | 0 | 0 |
| 10% Maltitol | 138* | 7.46 | 143* | 3.70 | 111* | 6.12 | 6* | 1.07 |
| 15% Maltitol | 104 | 1.96 | 110* | 3.42 | 102* | 4.28 | 8* | 6.89 |
| 20% Maltitol | 107 | 3.84 | 116* | 7.28 | 113* | 7.04 | 7* | 2.30 |
| 25% Maltitol | 102 | 4.77 | 129* | 13.07 | 120* | 4.02 | 5* | 6.59 |

As demonstrated, DispersinB enzymatic activity largely remained around 100% when up to 25% of maltitol by weight was added to the composition and incubated at 4° C. for 3 hours. DispersinB enzymatic activity largely remained increased over 100% when up to 25% of maltitol by weight was added to the composition and incubated at 42° C. for 3 hours. DispersinB enzymatic activity largely remained around 100% when 10% to 25% of maltitol by weight was added to the composition and incubated at 52° C. for 3 hours.

The clinically relevant maintenance of DispersinB enzymatic activity with the use of maltitol indicates that maltitol is useful in stabilizing DispersinB in liquid coating or film compositions at ambient or higher temperatures.

Thus, in one aspect, the present invention provides a use of maltitol with DispersinB in a liquid coating or film composition, to stabilize the DispersinB at an ambient or higher temperature.

In an embodiment, the amount of maltitol used is up to 25% of the composition by weight. In a preferred embodiment, the amount of maltitol used is between 5% and 20% of the composition by weight. In another preferred embodiment, the amount of maltitol used is between 10% and 15% of the composition by weight. In a further preferred embodiment, the amount of maltitol used is about 10% of the composition by weight.

In another aspect, the present invention provides a liquid coating or film composition comprising maltitol and DispersinB.

In an embodiment, the amount of maltitol in the liquid coating or film composition is up to 25% of the composition by weight. In a preferred embodiment, the amount of maltitol in the liquid coating or film composition is between 10% and 15% of the composition by weight. In a further preferred embodiment, the amount of sorbitol in the liquid coating or film composition is about 10% of the composition by weight.

### Use of a Polymer with DispersinB

A number of polymers were tested to determine their effect on the thermal stability of DispersinB at elevated temperatures. The polymers tested and their overall effect are set out in Table 13 below.

For each polymer, a DispersinB enzyme solution (100 μg/ml) was prepared in 50 mM citrate buffer (pH 5.9), 100 mM sodium chloride and polyols containing the above polymers. Samples from each formula were incubated at different temperatures room temperature or 42° C. for 20-24hrs hours.

Following incubation, DispersinB enzymatic activity of the samples was measured using β-N-Acetylglucosaminidase assay kit from Sigma (product code CS0780) in 96-well microtiter plate following the manufacturer's instructions. The data was represented as % enzymatic activity in comparison to enzyme activity of freshly made control sample that contained 100 μg/ml DispersinB in 50 mM citrate buffer (pH 5.9), 100 mM sodium chloride without polymers. Activity of the control sample was considered 100%. Results are shown in Table 13.

TABLE 13

| | Effect on |
|---|---|
| Polymer | DispersinB Activity |
| Carrageenan | Inhibit |
| Guar gum | Inhibit |
| Xanthan gum | Inhibit |
| Chitosan | Inhibit |
| Alginate | Inhibit |
| Polyacrylic acid | Inhibit |
| Sodium Carboxy Ethyl cellulose | Neutral |
| Sodium Carboxy methyl cellulose | Neutral |
| polyvinylpyrrolidone | Neutral |
| Poloxamer (Pluronic) | Enhance |
| Hydroxypropyl cellulose | Enhance |
| Hydroxypropyl Methyl cellulose | Enhance |
| Hydroxy Ethyl cellulose | Enhance |
| Gelatine | Enhance |
| Polyvinyl alcohol | Enhance |

Table 14 illustrates the effect of various polymers on thermal stability of DispersinB. * indicates statistically significant (p<0.05) values in paired two tailed t-test, with each treatment compared to the standard treatment containing 0% polymer.

TABLE 14

| | | RT 24 hrs | | 42° C. 20 hrs | |
|---|---|---|---|---|---|
| Polymer | % polymer | Mean | SD | Mean | SD |
| Polyvinyl alcohol | 2 | 149* | 5.93 | 127* | 6.76 |
| Polyvinyl alcohol | 1 | 154* | 7.59 | 130* | 1.28 |
| Polyvinyl alcohol | 0.5 | 140* | 2.59 | 125* | 6.38 |
| Polyvinyl alcohol | 0.25 | 135* | 4.08 | 126* | 5.03 |
| carrageenan | 2 | 111 | 2.31 | 26* | 0.43 |
| carrageenan | 1 | 49 | 25.62 | 26 | 16.57 |
| carrageenan | 0.5 | 3* | 2.41 | 50 | 5.26 |
| carrageenan | 0.25 | 11* | 7.40 | 57* | 3.56 |
| Guar gum | 1 | 114 | 14.15 | 128* | 6.93 |
| Guar gum | 0.5 | 83 | 7.49 | 81* | 4.83 |
| Guar gum | 0.25 | 70* | 4.06 | 63* | 1.07 |
| Guar gum | 0.125 | 96 | 4.27 | 80 | 4.63 |
| Xanthan gum | 1 | 67* | 6.19 | 50 | 14.75 |
| Xanthan gum | 0.5 | 73* | 4.37 | 49* | 3.08 |
| Xanthan gum | 0.25 | 57* | 1.61 | 39* | 2.52 |
| Xanthan gum | 0.125 | 86 | 4.51 | 62* | 2.68 |
| Sodium Carboxy Ethyl | 2 | 123 | 23.64 | 3* | 2.04 |
| cellulose | | | | | |
| Sodium Carboxy Ethyl | 1 | 92 | 7.56 | 7* | 0.77 |
| cellulose | | | | | |
| Sodium Carboxy Ethyl | 0.5 | 129 | 9.52 | 24* | 6.76 |
| cellulose | | | | | |
| Sodium Carboxy Ethyl | 0.25 | 115* | 3.87 | 100* | 3.11 |
| cellulose | | | | | |
| Sodium Carboxy methyl | 2 | 76* | 4.48 | 74 | 11.78 |
| cellulose | | | | | |
| Sodium Carboxy methyl | 1 | 105 | 3.71 | 74 | 7.44 |
| cellulose | | | | | |
| Sodium Carboxy methyl | 0.5 | 89 | 3.34 | 76* | 1.54 |
| cellulose | | | | | |
| Sodium Carboxy methyl | 0.25 | 100 | 7.98 | 81* | 1.67 |
| cellulose | | | | | |
| Hydroxypropyl cellulose | 2 | 154* | 5.94 | 127* | 2.64 |
| Hydroxypropyl cellulose | 1 | 134* | 4.01 | 118* | 3.23 |
| Hydroxypropyl cellulose | 0.5 | 123* | 2.13 | 124* | 5.56 |
| Hydroxypropyl cellulose | 0.25 | 122* | 0.89 | 124* | 5.34 |
| Hydroxypropyl cellulose | 0.125 | 113* | 0.58 | 116* | 4.63 |
| Hydroxypropyl cellulose | 0.0625 | 114* | 0.87 | 111* | 2.52 |
| Hydroxypropyl Methyl | 2 | 132* | 7.13 | 128 | 0.08 |
| cellulose | | | | | |
| Hydroxypropyl Methyl | 1 | 119* | 0.59 | 115 | 0.08 |
| cellulose | | | | | |
| Hydroxypropyl Methyl | 0.5 | 120* | 2.35 | 114 | 0.16 |
| cellulose | | | | | |
| Hydroxypropyl Methyl | 0.25 | 121* | 1.79 | 113 | 0.14 |
| cellulose | | | | | |
| Hydroxypropyl Methyl | 0.125 | 121* | 3.56 | 122* | 0.01 |
| cellulose | | | | | |
| Hydroxypropyl Methyl | 0.0625 | 116* | 6.54 | 125* | 0.01 |
| cellulose | | | | | |
| Hydroxy Ethyl cellulose | 2 | 125* | 10.99 | 101* | 12.89 |
| Hydroxy Ethyl cellulose | 1 | 119* | 6.07 | 88* | 7.37 |
| Hydroxy Ethyl cellulose | 0.5 | 120* | 6.86 | 89* | 4.09 |
| Hydroxy Ethyl cellulose | 0.25 | 117* | 3.16 | 90* | 4.64 |
| Sodium alginate | 2 | 25* | 1.90 | 0* | 1.28 |
| Sodium alginate | 1 | 44* | 2.80 | 9* | 0.93 |
| Sodium alginate | 0.5 | 52* | 2.63 | 30* | 3.64 |
| Sodium alginate | 0.25 | 68* | 5.75 | 42* | 0.93 |
| Gelatine (porcine) | 0.5 | 157* | 7.71 | 133* | 1.13 |
| Gelatine (porcine) | 0.25 | 165* | 9.80 | 140* | 2.90 |
| Gelatine (porcine) | 0.125 | 145* | 7.70 | 125* | 5.98 |
| Gelatine (porcine) | 0.0625 | 141* | 7.27 | 115* | 3.72 |
| polyvinylpyrrolidone | 2 | 99 | 4.20 | 80* | 4.50 |
| polyvinylpyrrolidone | 1 | 115* | 4.08 | 95* | 1.23 |
| polyvinylpyrrolidone | 0.5 | 98 | 1.33 | 90* | 3.48 |
| polyvinylpyrrolidone | 0.25 | 108 | 7.45 | 93 | 2.38 |
| Polyacrylic acid | 2 | 3* | 2.60 | — | — |
| Polyacrylic acid | 1 | 7* | 1.07 | — | — |
| Polyacrylic acid | 0.5 | 78* | 6.17 | — | — |
| Polyacrylic acid | 0.25 | 83 | 3.66 | — | — |
| Chitosan | 0.5 | 64* | 10.46 | — | — |
| Chitosan | 0.25 | 8* | 2.55 | — | — |
| Chitosan | 0.125 | 3* | 3.24 | — | — |
| Chitosan | 0.0625 | 0* | 1.94 | — | — |
| Chitosan | 0.03625 | 1* | 2.14 | — | — |
| Chitosan | 0.018125 | 6* | 3.57 | — | — |
| Pluronic F127 | 8 | 110* | 2.95 | 71* | 0.64 |
| Pluronic F127 | 6 | 109* | 4.12 | 94* | 1.38 |
| Pluronic F127 | 4 | 107* | 1.40 | 95* | 0.47 |
| Pluronic F127 | 3 | 108* | 1.07 | 103* | 2.17 |
| Pluronic F127 | 2 | 114* | 2.60 | 112* | 0.81 |
| Pluronic F127 | 1 | 114* | 0.64 | 99* | 0.94 |

As demonstrated in Table 14, strongly anionic polymers such as alginate, Xanthan, Guar gum, Carrageenan, and strongly cationic polymers such Chitosan, has reduced DispersinB enzymatic activity. Neutral polymers are preferred over ionic polymers. Among the ionic polymers, weakly anionic polymers are preferred over strongly cationic or anionic polymers. Non-ionic polymers (such as Pluronic, polyvinyl alcohol, and gelatin) have contributed to significantly enhance enzymatic activity rather than thermal stability.

Non-ionic polymers (highlighted in red) are found to enhance DispersinB activity and also render thermal stability of DispersinB. Table 14B sets out the ionic nature of the polymers tested with DispersinB.

TABLE 14B

| | |
|---|---|
| Polyvinyl alcohol | Non-ionic |
| Pluronic | Non-ionic |
| Hydroxypropyl cellulose | Non-ionic |
| Carboxy Ethyl cellulose | Non-ionic |
| Hydroxyethyl cellulose | Non-ionic |
| Guar gum | Non ionic- weakly anionic |
| Xanthan gum | anionic |
| Polyacrylic acid | anionic |
| Carrageenan | anionic |
| Alginate | anionic |
| Polyacrylic acid | anionic |
| Carboxymethyl cellulose | anionic |
| Hydroxypropyl methyl cellulose | anionic |
| Chitosan | cationic |
| polyvinylpyrrolidone | Cationic |

Thus, of the polymers tested, poloxamer (Pluronic), polyvinyl alcohol, gelatin, hydroxyethyl cellulose, carboxymethyl cellulose, and polyvinylpyrrolidone are preferred. Thus, in one aspect, the present invention provides a use of poloxamer (Pluronic), polyvinyl alcohol, gelatin, hydroxyethyl cellulose, carboxymethyl cellulose, and/or polyvinylpyrrolidone with DispersinB in a liquid coating or film composition, to stabilize the DispersinB at an ambient or higher temperature.

In particular, the polymer that contributed to both thermal stability and enzymatic activity is Pluronic F127 (also referred to as poloxamer 407) which it has shown to increase the enzymatic activity at 42° C. This is an exception to other polymers tested. Therefore, poloxamer 407 is particularly preferred over other non-ionic polymers.

Poloxamer 407 is a non-ionic triblock copolymer consisting of a central hydrophobic block of polypropylene glycol flanked by two hydrophilic blocks of polyethylene glycol. Polaxamer 407 is commonly used for its surfactant properties, such as an emulsifying agent, or solubilizing agent in cosmetic and personal products. Also referred to by its tradename as Pluronic F127 or PF127, the chemical structure of poloxamer 407 is:

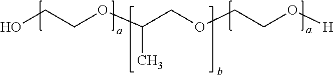

To test poloxamer 407, DispersinB enzyme solutions (100 μg/ml) were prepared in 50 mM citrate buffer (pH 5.9), 100 mM sodium chloride and 0-8% poloxamer 407. Samples from each formula was incubated at 3 different temperatures (42° C., 52° C. and 62° C.) for 3 hours. All the samples were then brought to room temperature for enzymatic activity assay.

DispersinB enzymatic activity was measured using β-N-Acetylglucosaminidase assay kit from Sigma (product code CS0780) in 96-well microtiter plate following the manufacturer's instructions. The data was represented as % enzymatic activity in comparison to enzyme activity of freshly made control sample that contained 100 μg/ml DispersinB in 50 mM citrate buffer (pH 5.9), 100 mM sodium chloride without poloxamer 407. Activity of the control sample was considered 100%.

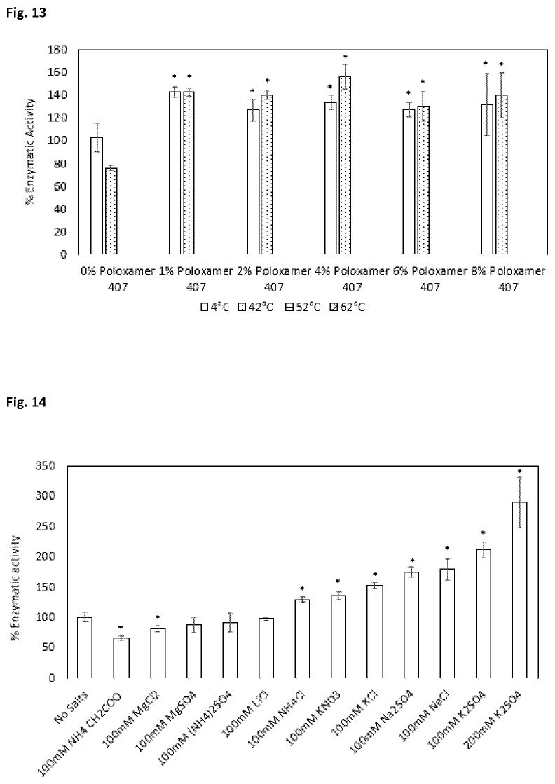

Table 15 and FIG. 13 illustrates the effect of poloxamer 407 on thermal stability of DispersinB. * indicates statistically significant (p<0.05) values in paired two tailed t-test, each treatment was compared with the DispersinB activity of a standard treatment containing no poloxamer 407.

TABLE 15

| | 4° C. | | 42° C. | | 52° C. | | 62° C. | |
|---|---|---|---|---|---|---|---|---|
| | Mean | SD | Mean | SD | Mean | SD | Mean | SD |
| PF127 0% | 103 | 12.26 | 76* | 2.28 | — | — | — | — |
| PF127 1% | 143* | 4.57 | 143* | 3.73 | — | — | — | — |
| PF127 2% | 127 | 9.645 | 140* | 3.95 | — | — | — | — |
| PF127 4% | 134* | 6.35 | 156* | 10.82 | — | — | — | — |
| PF127 6% | 127* | 6.37 | 130* | 12.29 | — | — | — | — |
| PF127 8% | 132* | 27.18 | 140* | 19.91 | — | — | — | — |

As demonstrated, the DispersinB enzymatic activity actually increased when up to 8% of poloxamer 407 was added to the composition and incubated at 42° C. for three hours. Notably, the DispersinB enzymatic activity increased significantly when when 4% of poloxamer 407 was added.

The clinically relevant maintenance of DispersinB enzymatic activity, with the use of poloxamer 407 indicates that poloxamer 407 is useful in stabilizing DispersinB in liquid coating or film compositions at an ambient or higher temperature.

Thus, in one aspect, the present invention provides a use of poloxamer 407 with DispersinB in a liquid coating or film composition, to stabilize the DispersinB at an ambient or higher temperature.

In an embodiment, the amount of poloxamer 407 used is up to 40 of the composition by weight. In a preferred embodiment, the amount of poloxamer 407 used is between 5% and 30% of the composition by weight. In another preferred embodiment, the amount of poloxamer 407 used is between 10% and 25% of the composition by weight. In a further preferred embodiment, the amount of poloxamer 407 used is about 16% of the composition by weight.

In another aspect, the present invention provides a liquid coating or film composition comprising poloxamer 407 and DispersinB.

In an embodiment, the amount of poloxamer 407 in the liquid coating or film composition is up to 10% of the composition by weight. In a preferred embodiment, the amount of poloxamer 407 in the liquid coating or film composition is between 1% and 8% of the composition by weight. In another preferred embodiment, the amount of poloxamer 407 in the liquid coating or film composition is between 3% and 5% of the composition by weight. In a further preferred embodiment, the amount of poloxamer 407 in the liquid coating or film composition is about 4% of the composition by weight.

### Use of Polymer in Erodible Systems

The polymers of the invention, set out herein, can be used to further stabilize Dispersin B in erodible systems, such as a polymer capsule or polymer matrix.

By an “erodible system” is meant an aqueous-erodible or water-swellable or aqueous-soluble in the sense of being either erodible or swellable or dissolvable (or combinations of these properties) in pure water or requiring the presence of an acid or base to ionize the polymeric matrix sufficiently to cause erosion or dissolution (e.g. gastric fluid). In other embodiments, the polymers for the erodible matrix comprises aqueous-soluble and aqueous-erodible cellulosics can include, for example, cellulose, methylethyl cellulose (MEC), carboxymethyl cellulose (CMC), CMEC, hydroxyethyl cellulose (HEC), hydroxypropyl cellulose (HPC), cellulose acetate (CA), cellulose propionate (CP), cellulose butyrate (CB), cellulose acetate butyrate (CAB), CAP, CAT, hydroxypropyl methyl cellulose (HPMC), HPMCP, IPMCAS, hydroxypropyl methyl cellulose acetate trimellitate (HPMCAT), and ethylhydroxy ethylcellulose (EHEC). In certain embodiments, the cellulosics comprises various grades of low viscosity (MW less than or equal to 50,000 Daltons, for example, the Dow Methocel™ series E5, E15LV, E5OLV and K100LY) and high viscosity (MW greater than 50,000 Daltons, for example, E4MCR, E1OMCR, K4M, K15M and K100M and the Methocel™ K series) HPMC. Other commercially available types of HPMC include the Shin Etsu Metolose 90SH series. Other materials useful as the erodible matrix material include, but are not limited to, pullulan, polyvinyl pyrrolidone (povidone), polyvinyl alcohol, polyvinyl acetate, glycerol fatty acid esters, polyacrylamide, polyacrylic acid, copolymers of ethacrylic acid or methacrylic acid (EUDRAGIT®, Rohm America, Inc., Piscataway, N.J.) and other acrylic acid derivatives such as homopolymers and copolymers of butylmethacrylate, methylmethacrylate, ethylmethacrylate, ethylacrylate, (2-dimethylaminoethyl) methacrylate, and (trimethylaminoethyl) methacrylate chloride.

### Use of Salt with DispersinB

Sodium chloride was used at a pH of 5.9 in DispersinB compositions when they are placed in long-term storage at −20° C. The present liquid coating or film composition may include a salt, which is a combination of cations: Na⁺, K⁺, Li⁺, Cs⁺, Ba²⁺, NH4⁺, Mn²⁺, Mg²⁺, Ca²⁺, Zn²⁺, Al³⁺; and anions: Cl⁻, NO₃⁻, SO₄²⁻, HPO₄²⁻CH2COOH⁻. Their concentrations may be in the range of 1 mM to 500 mM.

A number of salts were tested to determine their effect on the stability of DispersinB at ambient temperatures. The salts tested, and their overall effect, are set out in Table 16 below.

DispersinB enzyme solutions (100 μg/ml) were prepared in 50 mM citrate buffer, with pH 5.9 and containing 100-300 mM salts. DispersinB enzymatic activity was measured using β-N-Acetylglucosaminidase assay kit from Sigma (product code CS0780) in 96-well microtiter plate following the manufacturer's instructions. The data was represented as % enzymatic activity in comparison to enzyme activity of a control sample that contained 100 μg/ml DispersinB in 50 mM citrate buffer (pH 5.9), without salts. Activity of the control sample with no salts was considered 100%.

Table 16 and FIG. 14 illustrate the effect of salts on the enzymatic activity of DispersinB. * indicates statistically significant (p<0.05) values in paired two tailed t-test, each treatment was compared with enzymatic activity of DispersinB in a sample containing no salts.

TABLE 16

| Salt | Mean | SD |
|---|---|---|
| No salts | 100 | 7.50 |
| 100 mM NH4 CH2COO | 66* | 3.62 |
| 100 mM MgCl₂ | 81* | 5.5 |
| 100 mM MgSO₄ | 87 | 12.95 |
| 100 mM (NH₄)₂SO₄ | 91 | 15.15 |
| 100 mM LiCl | 97 | 3.35 |
| 100 mM NH₄Cl | 129* | 4.52 |
| 100 mM KNO₃ | 135* | 6.62 |
| 100 mM KCl | 152* | 4.99 |
| 100 mM Na₂SO₄ | 174* | 8.21 |
| 100 mM NaCl | 179* | 17.7 |
| 100 mM K₂SO₄ | 211* | 12.51 |
| 200 mM K₂SO₄ | 289* | 41.92 |

As demonstrated, NaCl, Na₂SO₄, NH₄Cl, KCl, KNO₃, and K₂SO₄ contributed to, and even enhanced, the enzymatic activity of DispersinB in a liquid coating or film composition. Thus, in one aspect, the present invention provides a use of NaCl, Na₂SO₄, NH₄Cl, KCl, KNO₃, and/or K₂SO₄ with DispersinB in a liquid coating or film composition, to stabilize the DispersinB. In particular, potassium sulfate (K₂SO₄) most significantly enhanced the enzymatic activity of DispersinB in a liquid coating or film composition.

Different pHs were then tested with potassium sulfate to determine if they had an effect on the stability of DispersinB.

DispersinB enzyme solutions (100 μg/ml) were prepared in 50 mM citrate buffer, containing 200 mM K₂SO₄. The pH was adjusted (5.4 to 5.9). DispersinB enzymatic activity was measured using β-N-Acetylglucosaminidase assay kit from Sigma (product code CS0780) in 96-well microtiter plate following the manufacturer's instructions. The data was represented as % enzymatic activity in comparison to enzyme activity of control sample that contained 100 μg/ml DispersinB in 50 mM citrate buffer (pH 5.9) containing 100 mM NaCl Activity of the control sample was considered 100%.

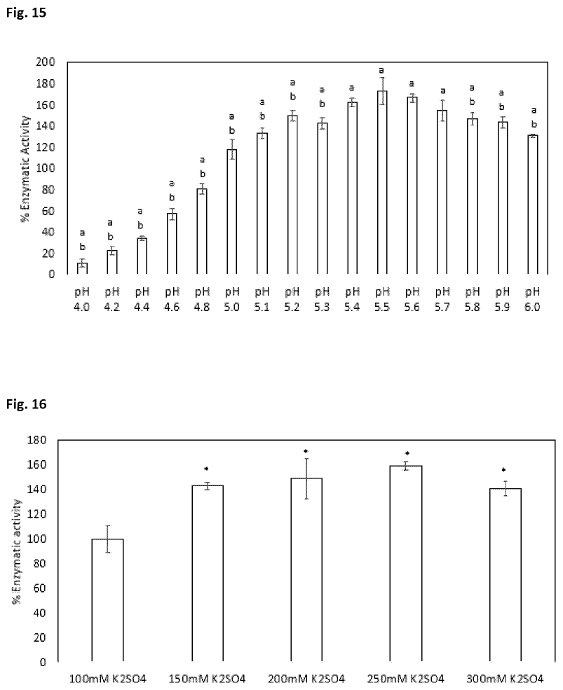

Table 17 and FIG. 15 illustrates the effect of pH on DispersinB activity in the presence of potassium sulfate.

TABLE 17

| Buffer | Salt | pH | % Activity | SD |
|---|---|---|---|---|
| 50 mM Citrate | 200 mM K₂SO₄ | 4 | 11.06^{ab} | 3.95 |
| 50 mM Citrate | 200 mM K₂SO₄ | 4.2 | 22.55^{ab} | 3.55 |
| 50 mM Citrate | 200 mM K₂SO₄ | 4.4 | 34.17^{ab} | 2.04 |
| 50 mM Citrate | 200 mM K₂SO₄ | 4.6 | 57.21 ^{ab} | 5.64 |
| 50 mM Citrate | 200 mM K₂SO₄ | 4.8 | 81.09 ^{ab} | 4.88 |
| 50 mM Citrate | 200 mM K₂SO₄ | 5 | 118.00 ^{ab} | 9.19 |
| 50 mM Citrate | 200 mM K₂SO₄ | 5.1 | 133.61 ^{ab} | 5.00 |
| 50 mM Citrate | 200 mM K₂SO₄ | 5.2 | 149.86 ^{ab} | 4.43 |
| 50 mM Citrate | 200 mM K₂SO₄ | 5.3 | 142.65 ^{ab} | 5.34 |
| 50 mM Citrate | 200 mM K₂SO₄ | 5.4 | 162.25^{a} | 3.80 |
| 50 mM Citrate | 200 mM K₂SO₄ | 5.5 | 172.83^{a} | 12.42 |
| 50 mM Citrate | 200 mM K₂SO₄ | 5.6 | 166.67^{a} | 3.96 |
| 50 mM Citrate | 200 mM K₂SO₄ | 5.7 | 154.62^{a} | 9.64 |
| 50 mM Citrate | 200 mM K₂SO₄ | 5.8 | 146.99 ^{ab} | 6.11 |
| 50 mM Citrate | 200 mM K₂SO₄ | 5.9 | 143.77 ^{ab} | 5.20 |
| 50 mM Citrate | 200 mM K₂SO₄ | 6 | 130.81 ^{ab} | 1.52 |
| 50 mM Citrate | 100 mM NaCl | 5.9 | 100.00^{b} | 6.53 |

“a” indicates statistically significant (p<0.05) values in paired two tailed t-test, each treatment compared with the enzymatic activity of DispersinB of sample containing 100 mM NaCl, pH 5.9.

“b” indicates statistically significant (p<0.05) values in paired two tailed t-test, each treatment compared with the enzymatic activity of DispersinB in sample containing 200 mM K2SO4, pH 5.5

As demonstrated in Table 17, enzymatic activity of DispersinB in the presence of potassium sulfate was most enhanced when the pH is around 5.5, rather than the traditional 5.9 with sodium chloride.

Different concentrations of potassium sulfate were also tested to determine if they had an effect on the stability of DispersinB in liquid coating or film compositions.

DispersinB enzyme solutions (100 μg/ml) were prepared in 50 mM citrate buffer (5.9), containing 100 mM to 300 mM K₂SO₄. DispersinB enzymatic activity was measured using β-N-Acetylglucosaminidase assay kit from Sigma (product code CS0780) in 96-well microtiter plate following the manufacturer's instructions. The data was represented as % enzymatic activity in comparison to enzyme activity of control sample that contained 100 μg/ml DispersinB in 50 mM citrate buffer (pH 5.9) 100 mM K₂SO₄. Activity of the control sample was considered 100%.

Table 18 and FIG. 16 illustrate the effect of potassium sulfate concentration on enzymatic activity of DispersinB. * indicates statistically significant (p<0.05) values in paired two tailed t-test, each treatment was compared with enzymatic activity of DispersinB of sample containing 100 mM K₂SO₄.

TABLE 18

| mM K₂SO₄ | % Activity | ±SD |
|---|---|---|
| 100 | 100 | 11.12 |
| 150 | 143* | 2.93 |
| 200 | 149* | 16.16 |
| 250 | 159* | 3.38 |
| 300 | 141* | 6.11 |

As demonstrated, enzymatic activity of DispersinB in the presence of potassium sulfate is notably enhanced when the concentration of potassium sulfate is above 100 mM, and especially when the potassium sulfate concentration is around 200 mM or 250 mM.

The clinically relevant maintenance of DispersinB enzymatic activity, with the use of potassium sulfate indicates that potassium sulfate is useful in stabilizing DispersinB in liquid coating or film compositions.

Thus, in one aspect, the present invention provides a use of a salt in a liquid coating or film composition to stabilize the DispersinB, where the salt is one of NaCl, Na₂SO₄, NH₄Cl, KCl, KNO₃, and K₂SO₄.

In an embodiment, the salt is potassium sulfate. In a preferred embodiment, the concentration of potassium sulfate used is up to 500 mM. In another preferred embodiment, the concentration of potassium sulfate used is between 100 and 400 mM. In a further preferred embodiment, the concentration of potassium sulfate used is between 200 and 300 mM. In a yet further preferred embodiment, the concentration of potassium sulfate is about 250 mM.

In the above embodiments of use of potassium sulfate with DispersinB, the liquid coating or film composition may have a pH between 5.2 and 5.9.

In another aspect, the present invention provides a liquid coating or film composition comprising a salt and DispersinB, where the salt is one of NaCl, Na₂SO4, NH₄Cl, KCl, KNO₃, and K₂SO₄.

In an embodiment, the salt is potassium sulfate. In a preferred embodiment, the concentration of potassium sulfate in the liquid coating or film composition is up to 500 mM. In another preferred embodiment, the concentration of potassium sulfate in the liquid coating or film composition is between 100 and 400 mM. In a further preferred embodiment, the concentration of potassium sulfate in the liquid coating or film composition is between 200 and 300 mM. In a yet further preferred embodiment, the concentration of potassium sulfate in the liquid coating or film composition is about 250 mM.

In the above embodiments of the liquid coating or film composition with potassium sulfate, the liquid coating or film composition may have a pH between 5.2 and 5.9. In a further preferred embodiment, the pH of the liquid coating or film composition is 5.9.

### Use of Preservative with DispersinB

DispersinB stock traditionally does not contain preservatives. Preservatives are generally not required because the DispersinB is typically either provided in lyophilized form, or it is stored at −20° C., so microbial growth is inhibited.

As well, when supplied to universities and companies, DispersinB solutions are often already sterilized, filtered, and mixed with heat sterilized glycerol. Then the end user would maintain the sterility of the DispersinB stock in order to avoid microbial growth at ambient or elevated temperatures.

Microbial growth may become an issue, however, when the liquid DispersinB composition is formulated, stored, and transported at ambient or elevated temperatures, and when the DispersinB is stored for long periods of time at those temperatures. Thus, a number of preservatives were tested to determine their effect on the stability of DispersinB, specifically, Levulinic acid (0.25%-2%), Anisic acid (0.3%), and EDTA (0.5%-10%). Of the antimicrobial compounds tested, it was found that certain concentrations of levulinic acid, anisic acid, and EDTA did not have an affect on DispersinB stability and enzymatic activity.

Levulinic acid, CH₃ C(O)CH₂CH₂CO₂H, or 4-oxopentanoic acid, is an organic compound classified as a keto acid. It is used as a precursor for pharmaceuticals, plasticizers, and other additives. For example, levulinic acid is commonly used in cosmetics, and as a precursor for biodegradable herbicides and fragrances/perfumes. The chemical structure of levulinic acid is:

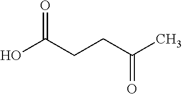

DispersinB enzyme (100 μg/ml) solutions in 50 mM citrate buffer (pH 5.9), 100 mM NaCl containing Levulinic acid (0.25%-2%) were tested for DispersinB enzymatic activity following β-N-Acetylglucosaminidase assay. The data was represented as % enzymatic activity in comparison to enzyme activity of freshly made control sample that contained 100 μg/ml DispersinB in 50 mM citrate buffer (pH 5.9), 100 mM sodium chloride. Activity of the control sample was considered 100%.

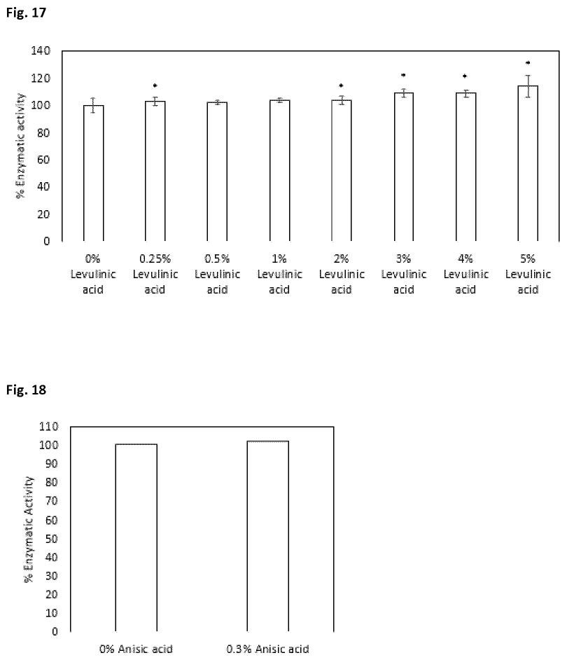

Table 19 and FIG. 17 illustrate the effect of levulinic acid on the enzymatic activity of DispersinB.* indicates statistically significant (p<0.05) values in paired two tailed t-test, where each treatment was compared with the enzymatic activity of DispersinB of a sample not containing Levulinic acid.

TABLE 19

| | Mean | ±SD |
|---|---|---|
| 0% Levulinic acid | 100 | 5.56 |
| 0.25% Levulinic acid | 103* | 3.33 |
| 0.5% Levulinic acid | 102 | 1.57 |
| 1% Levulinic acid | 104 | 1.57 |
| 2% Levulinic acid | 104* | 3.07 |
| 3% Levulinic acid | 109* | 3.23 |
| 4% Levulinic acid | 109* | 2.63 |
| 5% Levulinic acid | 114* | 7.88 |

As demonstrated, little to no change in DispersinB activity was detected with the use of levulinic acid.

The clinically relevant maintenance of DispersinB enzymatic activity with the use of levulinic acid indicates that levulinic acid is useful as a preservative in DispersinB liquid coating or film compositions.

Thus, in one aspect, the present invention provides a use of levulinic acid with DispersinB in a liquid coating or film composition to prevent microbial growth in DispersinB liquid coating or film compositions at an ambient or higher temperature.

In an embodiment, the concentration of levulinic acid used is up to 10%. In a preferred embodiment, the concentration of levulinic acid used is between 3% and 8%. In a further preferred embodiment, the concentration of levulinic acid used is about 5%.

In another aspect, the present invention provides a liquid coating or film composition comprising levulinic acid and DispersinB.

In an embodiment, the concentration of levulinic acid in the liquid coating or film composition is up to 10%. In a preferred embodiment, the concentration of levulinic acid in the liquid coating or film composition is between 3% and 8%. In a further preferred embodiment, the concentration of levulinic acid in the liquid coating or film composition is about 5%.

Anisic acid, C₈H8N₂O₃, or methoxybenzoic acid, is a carboxylic acid that may exist in one of three forms, p-Anisic acid, m-Anisic acid, or o-Anisic acid. Anisic acid has antiseptic properties, and it is often used as an intermediate in the preparation of more complex organic compounds. The chemical structure of anisic acid is:

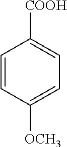 [SMILES: COc1ccc(C(=O)O)cc1]

DispersinB enzyme (100 μg/ml) solutions in 50 mM citrate buffer (pH 100 mM NaCl containing anisic acid (0.3%) were tested for DispersinB enzymatic activity following β-N-Acetylglucosaminidase assay. The data was represented as % enzymatic activity in comparison to enzyme activity of freshly made control sample that contained 100 μg/ml DispersinB in 50 mM citrate buffer (pH 5.9), 100 mM sodium chloride. Activity of the control sample was considered 100%.

Table 20 and FIG. 18 illustrate the effect of anisic acid on the enzymatic activity of DispersinB, where each treatment was compared with the enzymatic activity of DispersinB of a sample not containing anisic acid. “n.s” indicates that compared to the control group (0 mg/ml), the anisic acid concentration is not significantly different in a paired t-test.

TABLE 20

| Anisic acid | % activity | SD | p value | |
|---|---|---|---|---|
| 0% | 100 | 4.064 | | |
| 0.30% | 102 | 4.788 | 0.303 | n.s |

As demonstrated, little to no change in DispersinB activity was detected with the use of a lower concentration of anisic acid.

The clinically relevant maintenance of DispersinB enzymatic activity with the use of anisic acid indicates that anisic acid is useful as a preservative in DispersinB in liquid coating or film compositions.

Thus, in one aspect, the present invention provides a use of anisic acid with DispersinB in a liquid coating or film composition to prevent microbial growth in DispersinB liquid coating or film compositions at an ambient or higher temperature.

In an embodiment, the concentration of anisic acid used is about 0.3%.

In another aspect, the present invention provides a liquid coating or film composition comprising anisic acid and DispersinB.

In an embodiment, the concentration of anisic acid in the liquid coating or film composition is about 0.1% to 1%, preferably about 0.2% to 0.5%, more preferably about 0.3%.

Ethylenediaminetetraacetic acid (EDTA), C₁₀H₁₆N₂O₈, is a chemical used for both industrial and medical purposes. EDTA's usefulness arises because of its role as a hexadentate (“six-toothed”) ligand and chelating agent. The chemical structure of EDTA is:

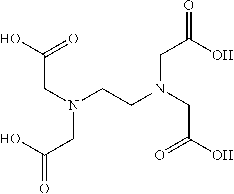 [SMILES: O=C(O)CN(CCN(CC(=O)O)CC(=O)O)CC(=O)O]

DispersinB enzyme (100 μg/ml) solutions in 50 mM citrate buffer and phosphate buffer (pH 5.9), with 100 mM NaCl containing EDTA (0.5%-10%) were tested for DispersinB enzymatic activity following β-N-Acetylglucosaminidase assay. The data was represented as % enzymatic activity in comparison to enzyme activity of freshly made control sample that contained 100 μg/ml DispersinB in 50 mM citrate buffer (pH 5.9), 100 mM sodium chloride. Activity of the control sample was considered 100%.

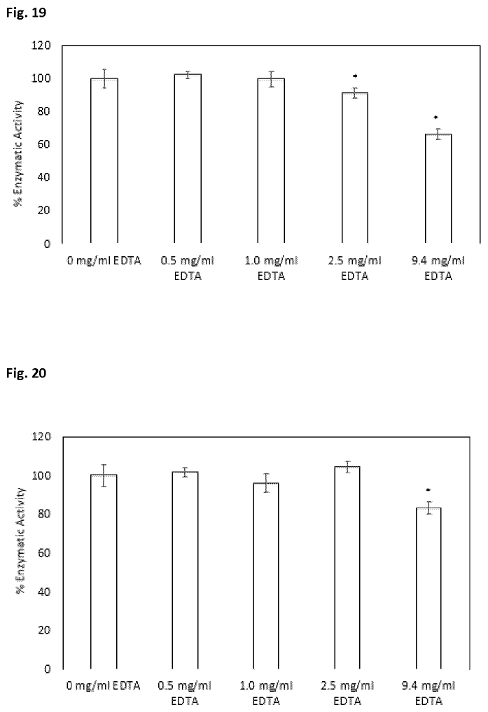

Tables 21 and 22 and FIGS. 19 and 20 illustrate the effect of EDTA on the enzymatic activity of DispersinB in the phosphate buffer. Table 21 illustrates the effect of EDTA on the enzymatic activity of DispersinB in the citrate buffer. “n.s” indicates that compared to control group (0 mg/ml), the EDTA concentration was not significantly different in paired t-test.

TABLE 21

| Buffer | EDTA | % | | | |
|---|---|---|---|---|---|
| System | (mg/ml) | Activity | SD | p valve | |
| Phosphate | 0 | 100.00 | 5.77 | | |
| Phosphate | 0.5 | 102.52 | 2.23 | 0.565 | n.s |
| Phosphate | 1 | 99.78 | 4.68 | 0.887 | n.s |
| Phosphate | 2.5 | 91.13 | 3.12 | 0.029 | |
| Phosphate | 9.4 | 66.51 | 3.29 | 0.005 | |

TABLE 22

| Buffer | EDTA | % | | | |
|---|---|---|---|---|---|
| System | (mg/ml) | Activity | SD | p valve | |
| Citrate | 0 | 100.00 | 5.81 | | |
| Citrate | 0.5 | 101.45 | 5.12 | 0.195 | n.s |
| Citrate | 1 | 95.86 | 4.67 | 0.169 | n.s |
| Citrate | 2.5 | 104.19 | 2.15 | 0.241 | n.s |
| Citrate | 9.4 | 83.18 | 1.90 | 0.018 | |

As demonstrated, the effect of EDTA concentration on DispersinB activity is slightly different in the two buffer systems. In citrate buffer, concentrations of EDTA that is greater than 2.5 mg/ml tends to reduce the enzyme activity. In the phosphate buffer, concentrations of EDTA that is greater than 1 mg/ml tends to reduce the enzyme activity if DispersinB. Thus, as demonstrated, there is little to no change in DispersinB activity with the use of EDTA that is less than 2.5 mg/ml in a citrate buffer, and with the use of EDTA that is less than 1 mg/ml in a phosphate buffer.

In addition, it was also found that not only does EDTA prevent microbial growth and destabilization of biofilm structure, EDTA also has the ability to chelate cations, such as iron, magnesium, and zinc.

The clinically relevant maintenance of DispersinB enzymatic activity with the use of EDTA indicates that EDTA is useful as a preservative in DispersinB liquid coating or film compositions.

Thus, in one aspect, the present invention provides a use of EDTA with DispersinB in a liquid coating or film composition with a citrate buffer to prevent microbial growth in DispersinB liquid coating or film compositions at an ambient or higher temperature.

In an embodiment, the concentration of EDTA used is up to 2.5%. In a preferred embodiment, the concentration of EDTA used is up to 1%. In a further preferred embodiment, the concentration of EDTA used is about 0.5%.

In another aspect, the present invention provides a liquid coating or film composition comprising EDTA, a citrate buffer, and DispersinB.

In an embodiment, the concentration of EDTA in the liquid coating or film composition is up to 2.5%. In a preferred embodiment, the concentration of EDTA n the liquid coating or film composition is up to 1%. In a further preferred embodiment, the concentration of EDTA n the liquid coating or film composition is about 0.5%.

### Use in Combination with DispersinB

### Polyol and Polymer in Combination

DispersinB in the traditional phosphate buffer, with sodium chloride at a pH of 5.9, is known to lose its enzymatic activity within 1 day at ambient temperature. DispersinB at an ambient or higher temperature was tested with a polyol and a polymer in combination.

In one example, sorbitol and poloxamer 407 were tested together to determine whether they collectively had an effect on the thermal stability of DispersinB B at elevated temperatures.

DispersinB enzyme solutions (100 μg/ml) were prepared in 50 mM citrate buffer (pH 5.9), 100 mM sodium chloride, 20-30% sorbitol and 0-8% poloxamer 407. Samples from each formula was incubated at 5 different temperatures; 4° C. and room temperature for 24 hours, and 42° C., 52° C. and 62° C. for 3 hours. All the samples were then brought to room temperature for enzymatic activity assay.

DispersinB enzymatic activity was measured using β-N-Acetylglucosaminidase assay kit from Sigma (product code CS0780) in 96-well microtiter plate following the manufacturer's instructions. The data was represented as % enzymatic activity in comparison to enzyme activity of freshly made control sample that contained 100 μg/ml DispersinB in 50 mM citrate buffer (pH 5.9), 100 mM sodium chloride with no sorbitol or poloxamer 407. Activity of the control sample was considered 100%.

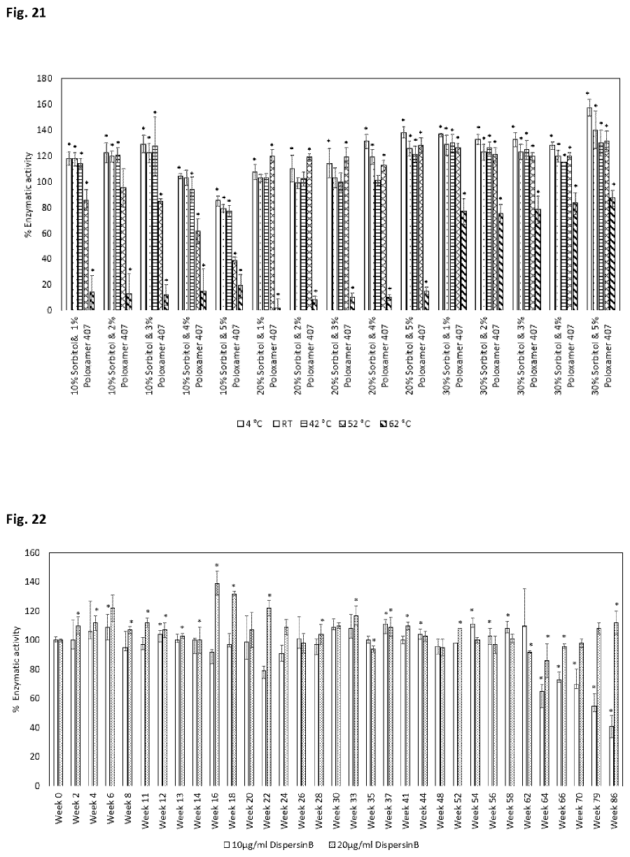

Table 23 and FIG. 21 illustrate the effect of sorbitol and poloxamer 407 on thermal stability and enzymatic activity of DispersinB. * indicates statistically significant (p<0.05) values in paired two tailed t-test, each treatment was compared with the DispersinB activity of standard treatment containing no sorbitol or poloxamer 407.

TABLE 23

| % | % | | | | | |
|---|---|---|---|---|---|---|
| Sorbitol | P407 | 4° C. | RT | 42° C. | 52° C. | 62° C. |
| 10 | 1 | 117* ± 4.99 | 117* ± 5.22 | 114* ± 3.47 | 85* ± 8.02 | 14* ± 13.46 |
| 10 | 2 | 122* ± 7.64 | 119* ± 4.06 | 120* ± 5.93 | 95 ± 15.15 | 13* ± 15.3 |
| 10 | 3 | 128* ± 6.75 | 121* ± 7.24 | 127* ± 22.55 | 84* ± 1.87 | 12* ± 8.03 |
| 10 | 4 | 104* ± 2.07 | 102 ± 5.55 | 93* ± 9.84 | 61* ± 9.71 | 15* ± 17.22 |
| 10 | 5 | 85* ± 3.88 | 79* ± 2.89 | 77* ± 4.75 | 38* ± 2.23 | 19* ± 9.15 |
| 20 | 1 | 107* ± 5.98 | 102 ± 2.8 | 102 ± 3.35 | 119* ± 5.02 | 2* ± 6.92 |
| 20 | 2 | 109* ± 10.32 | 98 ± 4.26 | 102 ± 5.41 | 118* ± 3.04 | 8* ± 3.1 |
| 20 | 3 | 114* ± 11.23 | 103 ± 7.58 | 99 ± 7.27 | 118* ± 7.47 | 10* ± 3.80 |
| 20 | 4 | 130* ± 5.59 | 119* ± 5.71 | 101 ± 3.88 | 112* ± 4.28 | 10* ± 2.05 |
| 20 | 5 | 137* ± 4.19 | 125* ± 5.6 | 121* ± 6.05 | 127* ± 5.85 | 15* ± 2.94 |
| 30 | 1 | 136* ± 1.2 | 128* ± 7.8 | 129* ± 6.77 | 125* ± 3.48 | 77* ± 10.02 |
| 30 | 2 | 132* ± 3.65 | 122* ± 6.31 | 125* ± 4.88 | 120* ± 5.41 | 75* ± 7.04 |
| 30 | 3 | 132* ± 5.42 | 122* ± 6.05 | 124* ± 7.56 | 119* ± 3.04 | 78* ± 10.47 |
| 30 | 4 | 127* ± 2.85 | 119* ± 4.56 | 114* ± 0.35 | 119* ± 2.61 | 83* ± 8.38 |
| 30 | 5 | 157* ± 6.35 | 139* ± 14.5 | 129* ± 9.86 | 131* ± 8.2 | 87* ± 5.97 |

As demonstrated, a combination of sorbitol and poloxamer 407 synergistically contributed to greater thermal stability and enzymatic activity of DispersinB than when sorbitol and poloxamer 407 were used individually. This was most clearly seen when the composition was incubated at 62° C. for three hours. A combination of 30% of sorbitol by weight and 5% of poloxamer 407 by weight resulted in particularly synergist thermal stability and higher enzymatic activity of DispersinB.

The clinically relevant maintenance of DispersinB enzymatic activity with the use of sorbitol and poloxamer 407 indicates that a polyol and a polymer in combination is useful in stabilizing DispersinB in liquid coating or film compositions at an ambient or higher temperature.

Thus, in one aspect, the present invention provides a use of a polyol and a polymer DispersinB in a liquid coating or film composition, to stabilize the DispersinB at an ambient or higher temperature.

In an embodiment, the polyol is sorbitol and the amount of sorbitol used is up to 50% of the composition by weight. In a preferred embodiment, the amount of sorbitol used is between 10 and 40% of the composition by weight. In a further preferred embodiment, the amount of sorbitol used is about 30% of the composition by weight.

In another embodiment, the polymer is poloxamer 407 and the amount of poloxamer 407 used is up to 10% of the composition by weight. In a preferred embodiment, the amount of poloxamer 407 used is between 4% and 6% of the composition by weight. In a further preferred embodiment, the amount of poloxamer 407 used is about 5% of the composition by weight.

In another aspect, the present invention provides a liquid coating or film composition comprising a polyol, a polymer, and DispersinB.

In an embodiment, the polyol is sorbitol and the amount of sorbitol in the liquid coating or film composition is up to 50% of the composition by weight. In a preferred embodiment, the amount of sorbitol in the liquid coating or film composition is between 10% and 40% of the composition by weight. In a further preferred embodiment, the amount of sorbitol in the liquid coating or film composition is about 30% of the composition by weight.

In another embodiment, the polymer is poloxamer 407 and the amount of poloxamer 407 in the liquid coating or film composition is up to 10% of the composition by weight. In a preferred embodiment, the amount of poloxamer 407 in the liquid coating or film composition is between 4% and 6% of the composition by weight. In a further preferred embodiment, the amount of poloxamer 407 in the liquid coating or film composition is about 5% of the composition by weight.

### Polyol, Polymer, and Preservatives in Combination

In another example, DispersinB at an ambient or higher temperature was tested with one or more of a polyol, a polymer, a buffering agent, a salt, and a preservative in combination.

In one test, DispersinB compositions containing 10-20 μg/ml of DispersinB, 30% sorbitol, 5% poloxamer 407, 50 mM Citrate buffer (pH 5.9), 100 mM sodium chloride, 1% Levulinic acid, 0.3% anisic acid, and 0.1% EDTA (1 mg/ml) were made and stored at room temperature, 40° C., and 45° C. The enzyme activity was measured using a β-N-Acetylglucosaminidase assay Kit (Sigma) at different time points.

Table 24 and FIG. 22 illustrate the enzymatic activity of DispersinB-10 and DispersinB-20 formulas at ambient or room temperature. “*” indicates statistically significant (p<0.05) values in paired two tailed t-test, where each treatment was compared with the sample stored at 4° C.

TABLE 24

| Weeks of | 10 μg/ml DispersinB | 20 μg/ml DispersinB |
|---|---|---|
| storage at | % Enzyme activity | % Enzyme activity |
| Room Temp | (Mean ± SD) | (Mean ± SD) |
| 0 | 100 ± 2.59 | 100 ± 1.15 |
| 2 | 100 ± 13.85 | 110* ± 5.8 |
| 4 | 106 ± 20.88 | 112* ± 5.04 |
| 6 | 109* ± 8.82 | 122* ± 8.99 |
| 8 | 95 ± 10.84 | 107* ± 2.32 |
| 11 | 97 ± 4.75 | 112* ± 3.35 |
| 12 | 104* ± 2.61 | 107* ± 5.2 |
| 13 | 100 ± 3.96 | 103* ± 1.66 |
| 14 | 100 ± 1.49 | 100* ± 8.95 |
| 16 | 92* ± 1.48 | 139* ± 8.15 |
| 18 | 97 ± 7.59 | 132* ± 1.62 |
| 20 | 99 ± 17.96 | 107 ± 11.89 |
| 22 | 79 ± 3.31 | 122* ± 5.31 |
| 24 | 91 ± 5.8 | 109 ± 5.33 |
| 26 | 101 ± 14.82 | 98 ± 6.43 |
| 28 | 97 ± 3.86 | 104* ± 6.78 |
| 30 | 109 ± 5.73 | 110 ± 1.93 |
| 33 | 108 ± 9.81 | 117* ± 6.59 |
| 35 | 100 ± 2.68 | 94* ± 2.03 |
| 37 | 111* ± 3.23 | 109* ± 6.51 |
| 41 | 100 ± 3.04 | 110* ± 2.66 |
| 44 | 104* ± 3.75 | 103 ± 3.55 |
| 48 | 96 ± 4.42 | 95 ± 5.69 |
| 52 | 98 ± 0 | 108* ± 0 |
| 54 | 111* ± 4.02 | 100 ± 1.84 |
| 56 | 103* ± 5.24 | 97 ± 5.92 |
| 58 | 108* ± 5.1 | 101 ± 3 |
| 62 | 110 ± 25.43 | 92* ± 0.77 |
| 64 | 65* ± 4.67 | 86* ± 11.63 |
| 66 | 73* ± 4.99 | 96* ± 1.74 |
| 70 | 70* ± 10.27 | 98 ± 3.15 |
| 79 | 55* ± 8.24 | 108 ± 3.96 |
| 86 | 41* ± 7.42 | 112* ± 8.05 |

For compositions with 10 μg/ml and 20 μg/ml of DispersinB, the compositions retained at least 90% of their initial enzymatic activity for at least 62 weeks, and at least 50% of their initial enzymatic activity for at least 79 weeks at ambient temperature.

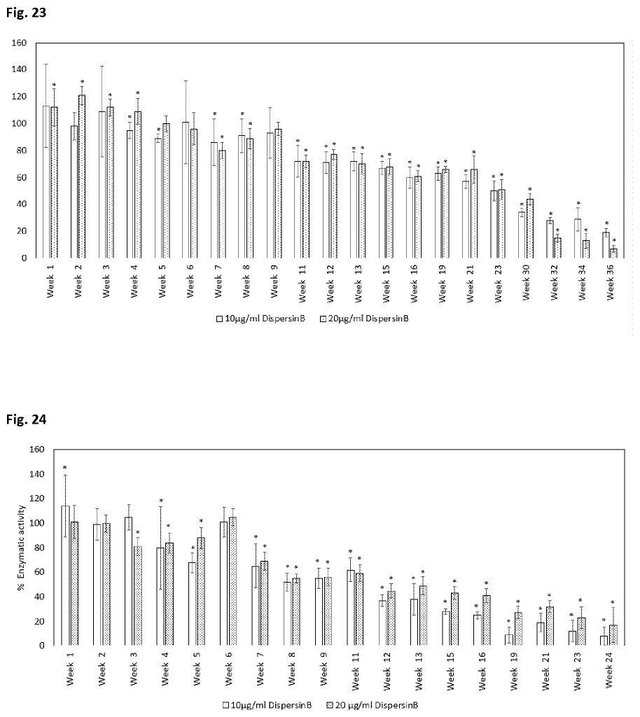

Table 25 and FIG. 23 illustrates the enzymatic activity of DispersinB-10 and DispersinB-20 formulas at 40° C. “*” indicates statistically significant (p<0.05) values in paired two tailed t-test, where each treatment was compared with the sample stored at 4° C.

TABLE 25

| Weeks of | 10 μg/ml DispersinB | 20 μg/ml DispersinB |
|---|---|---|
| storage | % Enzyme activity | % Enzyme activity |
| at 40° C. | (Mean ± SD) | (Mean ± SD) |
| 1 | 113 ± 30.92 | 112* ± 13.68 |
| 2 | 98 ± 10.16 | 121* ± 6.82 |
| 3 | 109 ± 33.62 | 112* ± 6.13 |
| 4 | 95* ± 5.96 | 109* ± 9.26 |
| 5 | 89* ± 3.14 | 100 ± 5.9 |
| 6 | 101 ± 30.93 | 96 ± 11.93 |
| 7 | 86* ± 17.42 | 80* ± 5.9 |
| 8 | 91* ± 12.43 | 89* ± 7.39 |
| 9 | 93 ± 18.54 | 96 ± 4.81 |
| 11 | 72* ± 11.67 | 72* ± 4.68 |
| 12 | 71* ± 7.99 | 77* ± 3.76 |
| 13 | 72* ± 7.1 | 70* ± 7.86 |
| 15 | 67* ± 4.82 | 68* ± 5.79 |
| 16 | 60* ± 7.91 | 61* ± 4.05 |
| 19 | 63* ± 5.05 | 66* ± 2.47 |
| 21 | 57* ± 5.23 | 66* ± 10.28 |
| 23 | 50* ± 7.23 | 51* ± 7.65 |
| 30 | 34* ± 3.05 | 44* ± 4.12 |
| 32 | 28* ± 2.36 | 15* ± 2.7 |
| 34 | 29* ± 8.63 | 13* ± 5.42 |
| 36 | 19* ± 3.04 | 7* ± 2.36 |

As demonstrated, at 40° C., the enzyme composition retained at least 90% of its initial enzymatic activity for at least 9 weeks, and at least 50% of initial enzymatic activity for at least 22 weeks at 40° C.

Table 26 and FIG. 24 illustrate the enzymatic activity of DispersinB-10 and DispersinB-20 formulas at 45° C. “*” indicates statistically significant (p<0.05) values in paired two tailed t-test, where each treatment was compared with the sample stored at 4° C.

TABLE 26

| Weeks of | 10 μg/ml DispersinB | 20 μg/ml DispersinB |
|---|---|---|
| storage at | % Enzyme activity | % Enzyme activity |
| 45° C. | (Mean ± SD) | (Mean ± SD) |
| 1 | 114* ± 25.43 | 101 ± 13.49 |
| 2 | 99 ± 13.02 | 100 ± 7.02 |
| 3 | 105 ± 10.35 | 81* ± 7.45 |
| 4 | 80* ± 33.67 | 84* ± 7.95 |
| 5 | 68* ± 8.04 | 88* ± 8.35 |
| 6 | 101 ± 12.24 | 105 ± 7.1 |
| 7 | 65* ± 17.85 | 69* ± 7.28 |
| 8 | 52* ± 7.14 | 55* ± 3.52 |
| 9 | 55* ± 8.22 | 56* ± 7.23 |
| 11 | 62* ± 9.4 | 59* ± 6.75 |
| 12 | 37* ± 4.89 | 45* ± 5.85 |
| 13 | 38* ± 12.77 | 49* ± 7.39 |
| 15 | 28* ± 2.46 | 43* ± 5.13 |
| 16 | 25* ± 2.91 | 41* ± 5.78 |
| 19 | 9* ± 6.45 | 27* ± 5.18 |
| 21 | 19* ± 7.42 | 32* ± 4.77 |
| 23 | 12* ± 8.87 | 23* ± 8.62 |
| 24 | 8* ± 7.04 | 17* ± 14.17 |

As demonstrated, at 45° C., the enzyme composition retained at least 90% of its initial enzymatic activity for at least 3 weeks, and at least 50% of initial enzymatic activity for at least 9 weeks at 45° C.

In another test, the stability of DispersinB compositions were measured by biofilm dispersal. DispersinB compositions containing 10 μg/ml DispersinB, 30% sorbitol, 5% PF127, 50 mM citrate buffer (pH 5.9), 100 mM sodium chloride, 1% levulinic acid, 0.3% anisic acid, and 0.1% EDTA were tested. Formulations of the same composition that were devoid of DispersinB was used as negative control in the experiments.

The formulations were stored at room temperature and used to test biological activity of DispersinB by biofilm dispersal assay at monthly intervals using overnight grown E. coli TRMG 1655, and methicillin-resistant S. pseudintermedius (MRSP) biofilms. Biofilms were grown in 96 well microtiter plates for 20 hours at 37° C., washed with distilled water, treated with DispersinB composition containing DispersinB for 5 minutes at 37° C., then washed and stained with crystal violet. The unbound crystal violet was washed away, the biofilm bound crystal violet was dissolved in ethanol acetic acid solution, and absorbance was measured at 620 nm. The absorbance value is the quantitative measurement of remaining biofilm. The absorbance value of the DispersinB untreated biofilm was considered 100%, and the remaining biofilm of DispersinB treated was represented as a % in comparison to DispersinB untreated.

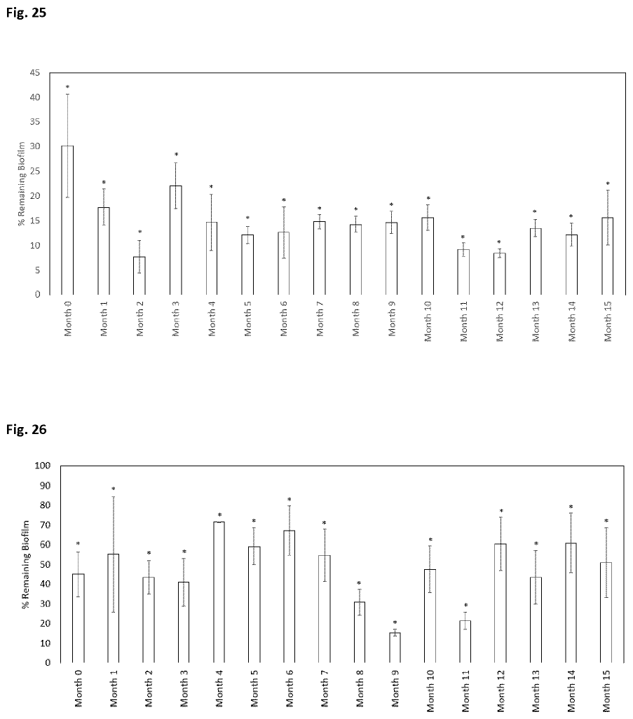

Table 27 and FIG. 25 illustrate the biofilm dispersal activity of the DispersinB compositions stored at room temperature on E. coli Biofilms. “*” indicates statistically significant (p<0.05) values in paired two tailed t-test, each treatment was compared with the DispersinB untreated control which was considered as 100% biofilm.

TABLE 27

| Months of | % of remaining biofilm |
|---|---|
| Storage at RT | (Mean ± SD) |
| 0 | 30.19* ± 10.37 |
| 1 | 17.76* ± 3.68 |
| 2 | 7.71* ± 3.28 |
| 3 | 22.07* ± 4.68 |
| 4 | 14.70* ± 5.72 |
| 5 | 12.13* ± 1.76 |
| 6 | 12.65* ± 5.22 |
| 7 | 14.86* ± 1.49 |
| 8 | 14.26* ± 1.59 |
| 9 | 14.64* ± 2.23 |
| 10 | 15.67* ± 2.58 |
| 11 | 9.20* ± 1.35 |
| 12 | 8.42* ± 0.92 |
| 13 | 13.49* ± 1.72 |
| 14 | 12.19* ± 2.32 |
| 15 | 15.60* ± 5.53 |

Table 28 and FIG. 26 illustrate the biofilm dispersal activity of DispersinB composition stored at room temperature on methicillin-resistant S. pseudintermedius (MRSP) biofilms. “*” indicates statistically significant (p<0.05) values in paired two tailed t-test, each treatment was compared with the DispersinB untreated control which was considered as 100% biofilm.

TABLE 28

| Months of | % of remaining biofilm |
|---|---|
| Storage at RT | (Mean ± SD) |
| 0 | 45.06* ± 11.23 |
| 1 | 55.25* ± 29.24 |
| 2 | 43.49* ± 8.32 |
| 3 | 41.02* ± 11.99 |
| 4 | 71.44* ± 0.06 |
| 5 | 59.15* ± 9.24 |
| 6 | 67.17* ± 12.47 |
| 7 | 54.71* ± 13.2 |
| 8 | 30.82* ± 6.51 |
| 9 | 15.38* ± 1.63 |
| 10 | 47.67* ± 11.82 |
| 11 | 21.37* ± 4.42 |
| 12 | 60.44* ± 13.49 |
| 13 | 43.63* ± 13.56 |
| 14 | 60.87* ± 15.09 |
| 15 | 50.94* ± 17.49 |

As demonstrated, in both cases, the biofilm dispersal activity remained largely stable over 15 months. Since there is more than 50% reduction (in most cases) of biofilm upon DispersinB treatment, the data points are statistically significant.

The clinically relevant maintenance of DispersinB enzymatic activity at ambient or higher temperatures over long periods of time with the use of a polyol, a polymer, and preservatives indicate that such combinations is useful as long term stabilizers of DispersinB liquid coating or film compositions. Biofilm dispersal activity is also shown to be sustained over an extended period of time.

Thus, in one aspect, the present invention provides a use of a polyol, a polymer, and a preservative with DispersinB in a liquid coating or film composition to stabilize and sterilize the DispersinB at an ambient or higher temperature.

In an embodiment, the preservative may be levulinic acid, anisic acid, or ethylenediaminetetraacetic acid (EDTA). In an alternate embodiment, the preservative may be a combination of levulinic acid, anisic acid, and ethylenediaminetetraacetic acid (EDTA). In embodiments where the preservative includes EDTA, a concentration of up to 2.5% of the EDTA is used. In an embodiment where the preservative includes EDTA, levulinic acid, and anisic acid, the concentration of levulinic acid is about 1%, the concentration of anisic acid is about 0.3%, and the concentration of EDTA is about 0.1%.

In another aspect, the present invention provides a liquid coating or film composition comprising DispersinB, a polyol, a polymer, and preservatives, wherein the presence of the polyol, polymer, and preservative stabilize and sterilize the composition at an ambient or higher temperature.

In an embodiment, the preservative may be levulinic acid, anisic acid, or ethylenediaminetetraacetic acid (EDTA). In an alternate embodiment, the preservative may be a combination of levulinic acid, anisic acid, and ethylenediaminetetraacetic acid (EDTA). In embodiments where the preservative includes EDTA, it has a concentration of up to 2.5%. In an embodiment where the preservative includes EDTA, levulinic acid, and anisic acid, the concentration of levulinic acid is about 1%, the concentration of anisic acid is about 0.3%, and the concentration of EDTA is about 0.1%.

### Applications

The present DispersinB containing liquid solutions, including aerosols, spays, gels, lotions, creams, and softgels may be manufactured, stored and transported at higher than refrigeration temperatures without losing enzymatic activity.

DispersinB enzymatic activity of compositions of the present invention also tend to be more stable at body temperatures of human and animals for longer than liquid coating or film compositions without polyols and polymers. They may, therefore, generally suitable for medical and cosmetic use. Present compositions may be used topically for skin care, wound care, oral care, optic care, ophthalmic care, nasal care, hair care, lung care, and as a general surface cleaning agent for dispersal of preformed biofilms and inhibition of biofilm formation.

Present compositions may also be used internally as a coating on medical devices, intravenous injections, on surgical sites to prevent biofilm formation, and disperse preformed biofilms.

Since the present DispersinB compositions tend to be stable at body temperatures and maintain biofilm dispersal activity for a long period of time, the present uses and compositions may also be used as slow or fast release soft gel for oral or rectal use to prevent biofilm formation and disperse preformed biofilms of the digestive system.

Present compositions may also include additional ingredients such thickening agents to maintain desired viscosity, and colouring and fragrances to improve user appeal. The additives should not affect DispersinB stability and activity at recommended concentrations.

The amount of DispersinB in the composition can be in the range of 1-5000 μg/ml. The preferred concentration is 10-200 μg/ml. The enhanced stability of the present DispersinB composition allows smaller amounts of DispersinB to be used in the compositions.

Throughout the description, specific details are set forth in order to provide a more thorough understanding to persons skilled in the art. However, well known elements may not have been shown or described in detail to avoid unnecessarily obscuring the disclosure. Accordingly, the description and drawings are to be regarded in an illustrative, rather than a restrictive, sense.

While a number of exemplary aspects and embodiments have been discussed above, those of skilled in the art will recognize certain modifications, permutations, additions and sub-combinations thereof. It is therefore intended that the following appended claims and claims hereafter introduced are interpreted to include all such modifications, permutations, additions and sub-combinations as are consistent with the broadest interpretation of the specification as a whole.

What is claimed is:

1. (canceled)

2. (canceled)

3. (canceled)

4. (canceled)

5. (canceled)

6. (canceled)

7. (canceled)

8. (canceled)

9. (canceled)

10. (canceled)

11. (canceled)

12. (canceled)

13. (canceled)

14. Use of a polyol in a liquid coating or film composition with DispersinB to stabilize the DispersinB at an ambient or higher than ambient temperature, the polyol comprising one or more of sorbitol, propylene glycol, inositol, isomalt, erythritol, and maltitol.

15. A composition comprising: DispersinB and a polyol, wherein the presence of the polyol in the composition stabilizes the DispersinB at an ambient or higher than ambient temperature, the polyol comprising one or more of sorbitol, propylene glycol, inositol, isomalt, erythritol, and maltitol.

16. The use of claim 14, wherein the polyol is sorbitol optionally wherein the amount of sorbitol is one or more of up to 50% of the composition by weight, between 5% and 50% of the composition by weight, between 25% and 35% of the composition by weight, and about 30% of the composition by weight.

17. (canceled)

18. (canceled)

19. (canceled)

20. (canceled)

21. The composition of claim 14, wherein the polyol is sorbitol, optionally wherein the amount of sorbitol is one or more of up to 50% of the composition by weight, between 5% and 50% of the composition by weight, between 15% and 30% of the composition by weight, and about 20% of the composition by weight.

22. (canceled)

23. (canceled)

24. (canceled)

25. (canceled)

26. The use of claim 14, wherein the polyol is propylene glycol, optionally wherein the amount of propylene glycol is one or more of up to 40% of the composition by weight, between 1% and 30% of the composition by weight, and about 2.5% of the composition by weight.

27. (canceled)

28. (canceled)

29. (canceled)

30. The composition of claim 15, wherein the polyol is propylene glycol, optionally wherein the amount of propylene glycol is one or more of up to 40% of the composition by weight, between 1% and 30% of the composition by weight, and about 2.5% of the composition by weight.

31. (canceled)

32. (canceled)

33. (canceled)

34. The use of claim 14, wherein the polyol is inositol, optionally wherein the amount of inositol is one or more of up to 25% of the composition by weight, between 10% and 20% of the composition by weight, and about 14% of the composition by weight.

35. (canceled)

36. (canceled)

37. (canceled)

38. The composition of claim 15, wherein the polyol is inositol, optionally wherein the amount of inositol is one or more of up to 25% of the composition by weight, between 10% and 20% of the composition by weight, and about 14% of the composition by weight.

39. (canceled)

40. (canceled)

41. (canceled)

42. The use of claim 14, wherein the polyol is isomalt, optionally wherein the amount of isomalt is one or more of up to 20% of the composition by weight, between 1% and 20% of the composition by weight, and about 1% of the composition by weight.

43. (canceled)

44. (canceled)

45. (canceled)

46. The composition of claim 15, wherein the polyol is isomalt, optionally wherein the amount of isomalt is one or more of up to 20% of the composition by weight, between 1% and 20% of the composition by weight, and about 1% of the composition by weight.

47. (canceled)

48. (canceled)

49. (canceled)

50. The use of claim 14, wherein the polyol is erythritol, optionally wherein the amount of erythritol is one or more of up to 25% of the composition by weight, between 1% and 20% of the composition by weight, and about 25% of the composition by weight.

51. (canceled)

52. (canceled)

53. (canceled)

54. The composition of claim 15, wherein the polyol is erythritol, optionally wherein the amount of erythritol is one or more of up to 25% of the composition by weight, between 1% and 20% of the composition by weight, and about 25% of the composition by weight.

55. (canceled)

56. (canceled)

57. (canceled)

58. The use of claim 14, wherein the polyol is maltitol, optionally wherein the amount of maltitol is up to 25% of the composition by weight, between 1% and 25% of the composition by weight, between 10% and 25% of the composition by weight and about 10% of the composition by weight.

59. (canceled)

60. (canceled)

61. (canceled)

62. (canceled)

63. The composition of claim 15, wherein the polyol is maltitol, optionally wherein the amount of maltitol is up to 25% of the composition by weight, between 1% and 25% of the composition by weight, between 10% and 25% of the composition by weight, and about 10% of the composition by weight.

64. (canceled)

65. (canceled)

67. (canceled)

68. (canceled)

69. (canceled)

70. (canceled)

71. (canceled)

72. (canceled)

73. (canceled)

74. (canceled)

75. (canceled)

76. (canceled)

77. (canceled)

78. (canceled)

79. (canceled)

80. (canceled)

81. (canceled)

82. (canceled)

83. (canceled)

84. (canceled)

85. (canceled)

86. (canceled)

87. (canceled)

88. (canceled)

89. (canceled)

90. (canceled)

91. (canceled)

92. (canceled)

93. (canceled)

94. (canceled)

95. (canceled)

96. (canceled)

97. (canceled)

98. (canceled)

99. (canceled)

100. (canceled)

101. (canceled)

102. (canceled)

103. (canceled)

104. (canceled)

105. (canceled)

106. (canceled)

107. (canceled)

108. (canceled)

109. (canceled)

110. (canceled)

111. (canceled)

112. (canceled)

113. (canceled)

114. (canceled)

115. (canceled)

116. The use of claim 14 further comprising a polymer in a liquid coating or film composition with the DispersinB to stabilize the DispersinB at an ambient or higher than ambient temperature,
the polymer comprising one or more of poloxamer 407, polyvinyl alcohol, cellulose polymers, hydroxyethyl cellulose, carboxymethyl cellulose, pluronic, hydroxypropyl cellulose, carboxy ethyl cellulose, and polyvinylpyrrolidone.

117. (canceled)

118. (canceled)

119. (canceled)

120. (canceled)

121. The use of claim 116, wherein the polymer is poloxamer 407, optionally wherein amount of poloxamer 407 is one or more of up to 10% of the composition by weight between 4% and 6% of the composition by weight and about 5% of the composition by weight.

122. (canceled)

123. (canceled)

124. (canceled)

125. The use of claim 116, further comprising use of a preservative, wherein the preservative is one or more of ethylenediaminetetraacetic acid (EDTA), levulinic acid, and anisic acid optionally wherein the preservative comprises ETDA with a concentration of up to 2.5% of the composition by weight, optionally wherein the concentration of levulinic acid is about 1%, the concentration of anisic acid is about 0.1%, and the concentration of EDTA is about 0.1% of the composition by weight.

126. (canceled)

127. (canceled)

127. (canceled)

128. (canceled)

129. (canceled)

130. A liquid coating or film comprising the composition of claim 15 further comprising: a polymer wherein the presence of the polyol and the polymer in the composition stabilize the DispersinB at an ambient or higher than ambient temperature
the polymer comprising one or more of poloxamer 407, polyvinyl alcohol, cellulose polymers, hydroxyethyl cellulose, carboxymethyl cellulose, pluronic, hydroxypropyl cellulose, carboxy ethyl cellulose, and polyvinylpyrrolidone.

131. The composition of claim 130, wherein the polyol is sorbitol, optionally wherein the amount of sorbitol is one or more of up to 40% of the composition by weight7 between 25% and 35% of the composition by weight, and about 30% of the composition by weight.

132. (canceled)

133. (canceled)

134. (canceled)

135. The composition of claim 130, wherein the polymer is poloxamer 407, optionally wherein amount of poloxamer 407 is one or more of up to 10% of the composition by weight7 between 4% and 6% of the composition by weight7 and about 5% of the composition by weight.

136. (canceled)

137. (canceled)

138. (canceled)

139. The composition of claim 130, further comprising a preservative, optionally wherein the preservative comprises one or more of levulinic acid, anisic acid, or ethylenediaminetetraacetic acid (EDTA). Optionally wherein the preservative comprises ETDA with a concentration of up to 2.5% of the composition by weight optionally wherein the concentration of levulinic acid is about 1%, the concentration of anisic acid is about 0.1%, and the concentration of EDTA is about 0.1% of the composition by weight.

140. (canceled)

141. (canceled)

142. (canceled)

143. (canceled)

144. (canceled)

145. (canceled)

146. (canceled)

147. (canceled)

148. (canceled)

149. (canceled)

150. (canceled)

151. (canceled)

152. (canceled)

153. (canceled)

154. The composition claim 15 within an erodible polymer system, optionally wherein the erodible polymer system is a capsule or a matrix optionally wherein the erodible polymer is a neutral polymer.

155. (canceled)

156. (canceled)
